SEQUENCE LISTING (11 sequences)

SEQ ID NO: 1 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 243 aa, synthetic construct
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 3 - protein, 247 aa, synthetic construct
MVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHAVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHH

SEQ ID NO: 4 - protein, 247 aa, synthetic construct
MVCKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHAVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHH

SEQ ID NO: 5 - protein, 466 aa, synthetic construct
MGWSCIILFLVATATGVHSQMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 239 aa, synthetic construct
MGWSCIILFLVATATGVHSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 468 aa, synthetic construct
MGWSCIILFLVATATGVHSEVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 235 aa, synthetic construct
MGWSCIILFLVATATGVHSDIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 478 aa, synthetic construct
MGWSCIILFLVATATGVHSQVQLLESGGGLVQPGGSLRLSCAASGFTFSSYAMSWVRQAPGKGLEWASAISGSGGSTYYADSVEGRFTISRDNSKNTLYLQMNSLRAEDTAVYYCAKDTGYCGDDCYIKLIRGGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 238 aa, synthetic construct
MGWSCIILFLVATATGVHSDIVMTQSPLSLPVTLGQPASISCRSSQSLVYSDGNTFLNWFQQRPGQSPRRLIYQVSNRDSGVPDRFSGSGSGTDFTLKISRVEAEDVGVYYCMQGTHWPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 853 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADENLYFQGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0064921 A1
(43) Pub. Date: Feb. 27, 2025

(54) CHEMICALLY MODIFIED BACTERIAL PEPTIDOGLYCAN COMPOSITIONS AND USES THEREOF

(71) Applicants: CZ Biohub SF, LLC, San Francisco, CA (US); The Board of Trustees of the Leland Stanford Junior University, Stanford, CA (US)

(72) Inventors: Payton Anders-Benner Weidenbacher, Menlo Park, CA (US); Frances P. Rodriguez-Rivera, Jersey City, NJ (US); Carolyn R. Bertozzi, Menlo Park, CA (US); Peter S. Kim, Stanford, CA (US)

(21) Appl. No.: 18/724,466

(22) PCT Filed: Dec. 16, 2022
(86) PCT No.: PCT/US2022/081848

§ 371 Date: Jun. 26, 2024

Related U.S. Application Data
(60) Provisional application No. 63/294,135, filed on Dec. 28, 2021.

(51) Int. Cl. A61K 39/385; A61K 39/00; A61K 39/085; A61K 39/215; A61K 47/64; A61K 47/69

(52) U.S. Cl. CPC A61K 39/385; A61K 39/0011; A61K 39/085; A61K 39/215; A61K 47/646; A61K 47/6925; A61K 2039/575; A61K 2039/6087; A61K 2039/627; A61K 2039/64

(57) ABSTRACT

Provided are compositions and formulations comprising an isolated Staphylococcus aureus peptidoglycan sacculus comprising an azide-modified D-amino acid and an immunogenic polypeptide attached thereto. The composition can optionally also comprise an adjuvant. Also provided are methods for inducing an immune response in a subject using such compositions and formulations. Kits are also provided.

### CROSS-REFERENCE TO RELATED APPLICATION[S]

This application claims priority to, and the benefit of, co-pending U.S. Provisional Application No. 63/294,135 entitled “CHEMICALLY MODIFIED BACTERIAL PEPTIDOGLYCAN COMPOSITIONS AND USES THEREOF” and filed on Dec. 28, 2021, the contents of which are incorporated in their entirety by reference as if fully set forth herein.

### REFERENCE TO A SEQUENCE LISTING SUBMITTED AS XML VIA PATENT CENTER

The instant application contains a Sequence Listing that has been filed electronically in .xml format and is hereby incorporated by reference in its entirety. Said .xml copy, created on Dec. 15, 2022, is named “103182-1352319-007710WO-SL.xml” and is 16 kilobytes in size.

### BACKGROUND

Subunit vaccines, also referred to as protein vaccines, contain purified or recombinant subunit components derived from a particular pathogen, such as proteins or peptides, that have antigenic properties. In comparison to their whole-pathogen counterparts, subunit vaccines have minimal adverse effects, do not require complex storage or transport conditions, do not require production of large amounts of virus, and have large-scale manufacturing potential. However, subunit vaccines are often unable to trigger strong immune responses and require the application of nanotechnology or molecular adjuvants to boost the immunity. Subunit vaccines are rapidly degraded and cleared from the body and generally have low intrinsic immunogenic properties. Vaccine scaffolds and carrier proteins may increase the immunogenicity of subunit vaccines. Some examples of clinically utilized carrier proteins are hemocyanins such as keyhole limpet hemocyanin (KLH), bovine serum albumin (BSA) purified by fractionation of bovine plasma, diphtheria toxoid, and tetanus toxoid. However, commercial scale manufacturing involves isolation from keyhole limpets (Megathura crenulata) and is difficult. Due to its native origin, KLH adjuvants have the risk of bio-contamination by pathogens such as pathogenic blood ingredients, toxins, bacteria, including endotoxins produced thereby, as well as viruses.

A variety of vaccine nanoparticles have been tested, like inorganic metal-based nanoparticles and lipid formulations, but these inorganic methods are challenging to scale-up to the amount required for a global vaccination campaign. In addition, these inorganic nanoparticles are neither degraded nor effectively cleared from the body, potentially leading to long-term adverse effects.

As such, there is a need for technologies that improve the immune response of subunit vaccines. While alternative adjuvants and carrier approaches have been pursued with success, there is an unmet need for immunogenic, easily adaptable carriers that can be produced at scale.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on the creation by the inventors of compositions comprising bacterial PGN microparticle scaffolds for immunogenic polypeptides. The PGN microparticle is biodegradable and is capable of eliciting an immune response against attached immunogenic polypeptides similar to that of commonly used carrier proteins like KLH. The described PGN microparticles are highly stable and are a scalable subunit vaccine conjugation platform.

In one aspect, provided herein are compositions comprising an isolated Staphylococcus aureus peptidoglycan (PGN) sacculus bonded to an immunogenic polypeptide via a triazole moiety.

In some embodiments, the triazole moiety is a reaction product between an azide-modified D-amino acid on the PGN sacculus and a cycloalkyne moiety bonded to the immunogenic polypeptide via a linker moiety.

In some embodiments, the azide-modified D-amino acid is azido-D-alanine (azaDala).

In some embodiments, the linker moiety is a reaction product between an amino acid residue in the immunogenic polypeptide and a crosslinker reagent comprising the cycloalkyne moiety and a peptide-reactive handle.

In some embodiments, the cycloalkyne moiety is a cyclooctyne moiety. In some embodiments, the cyclooctyne moiety is dibenzocyclooctyne (DBCO).

In some embodiments, the peptide-reactive handle is a maleimide and the amino acid residue in the immunogenic polypeptide is a cysteine residue.

In some embodiments, the peptide-reactive handle is a N-hydroxysuccinimide moiety.

In some embodiments, the crosslinker reagent further comprising one or more ethylene glycol moieties. In some embodiments, the one or more ethylene glycol moieties comprise polyethylene glycol (PEG). In some embodiments, the PEG is PEG3, PEG4, or PEG8.

In some embodiments, the PGN sacculus is a peptidoglycan sacculus selected from Staphylococcus aureus strain ATCC 25923, ATCC 29213, SH1000, RN4220, or RN4220 (ΔTarO). In some embodiments, the PGN sacculus is a peptidoglycan sacculus from Staphylococcus aureus strain SH1000.

In some embodiments, the PGN sacculus is bonded to a plurality of different immunogenic polypeptides via triazole moieties.

In some embodiments, the immunogenic polypeptide is a viral protein, a bacterial protein, a fungal protein, or a protein expressed in a cancer cell.

In some embodiments, the immunogenic polypeptide is a SARS-CoV-2 Spike protein or a fragment thereof.

In some embodiments, the immunogenic polypeptide is a SARS-CoV-2 Spike protein receptor binding domain (RBD).

In some embodiments, the immunogenic polypeptide is a cancer neoantigen.

In some embodiments, the composition further comprises an adjuvant and/or a stabilizing agent.

In some embodiments, the stabilizing agent comprises nanoparticle hydrogel.

In some embodiments, the adjuvant and/or the stabilizing agent are conjugated to the PGN sacculus.

In some embodiments, the adjuvant comprises at least one of a Toll-like receptor (TLR) agonist and/or a T-cell epitope.

In other aspects, provided herein are formulations comprising any of the described compositions and a pharmaceutically acceptable excipient.

In some embodiments, formulations as described herein further comprise an adjuvant.

In another aspect, provided are methods of inducing an immune response in a subject, the method comprising administering to the subject a therapeutically effective amount of any of the provided formulations.

In some embodiments, the method elicits an antibody response in the subject.

In some embodiments, the method elicits a T cell response in the subject.

In some embodiments, the immunogenic polypeptide is a SARS-CoV-2 Spike protein or a fragment thereof, and wherein the formulation is administered in an amount capable of eliciting a protective immune response against the SARS-CoV-2 Spike protein in the subject. In some embodiments, the subject has a SARS-CoV-2 infection, is suspected of having a SARS-CoV-2 infection, or is at risk of exposure to SARS-CoV-2 infection.

In some embodiments, the immunogenic polypeptide is a protein expressed in a cancer cell, and wherein the formulation is administered in an amount capable of eliciting a protective immune response against a cancer. In some embodiments, the protective immune response comprises production of neutralizing antibodies against the cancer in the subject. In some embodiments, the subject has, has had, or is at risk of developing the cancer.

In some embodiments, in the provided methods, the composition is administered to the subject subcutaneously, intramuscularly, intravenously, intranasally, or orally.

In another aspect, provided are kits comprising any of the provided formulations packaged, for example, in a container and instructions for the administration thereof. In some embodiments, the provided kits may also include an adjuvant. In some embodiments, the provided kits may also include an applicator. In some embodiments of the provided kits, the composition is lyophilized. In some embodiments of the provided kits, the formulation is present in an effective amount, dosage unit, or plurality of dosage units.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1A-IC depict PGN microparticles incorporating unnatural D-aa residues without altering their structure according to aspects of this disclosure. FIG. 1A shows a general schematic for incorporation of unnatural D-aa into PGN (dark-shaded oval), followed by purification and antigen conjugation, and the chemical structure of PGN (light-shaded oval) highlighting the D-ala residue that is likely replaced by the unnatural D-aa residue, indicated by an X (right panel). The conjugation method is shown on the right where an antigen is depicted as a circle “clicked” onto the unnatural D-aa (an X, right panel). FIG. 1B shows a schematic of PGN microparticle purification from growing cells. This includes boiling in 1 M NaCl to burst the membrane and release a variety of intracellular components, followed by Benzonase treatment to digest RNA and DNA, trypsin treatment to digest PGN-bound and cellular proteins, and finally incubation with 1 M HCl to remove wall teicoic acids. FIG. 1C shows dynamic light scattering traces of purified PGN microparticles from S. aureus, B. subtills, or L. monocytogenes containing Wild Type (WT) PGN (circle), or PGN isolated after growth in broth containing additional D-ala (square) or alkDala (triangle).

FIGS. 2A-2B illustrate PGN microparticles stimulate macrophages and conjugate subunit vaccine antigens according to aspects of this disclosure. FIG. 2A shows results of RAW-Blue macrophage stimulation assays comparing isolated PGN microparticles with either Wild Type (WT) strain (dark-shaded bars on left) or grown with additional D-ala (medium-shaded bars on left) or alkDala (light-shaded bars on left). Four strains of bacteria were tested, L. monocytogenes (Lm), L. monocytogenes with an OatA mutation (Lm OatA), L. monocytogenes with a PdgA mutation (Lm PgdA), or S. aureus (Sa). Positive controls, LPS (lipopolysaccharide), MDP (Muramyl dipeptide), and iE-DAP(gamma-D-Glu-mDAP) are shown as unfilled bars on right and negative controls, cells with substrate, medium with substrate, and substrate alone, are shown as filled bars on right. Mean and standard deviation is shown for an n=3, where each point is the mean of a triplicate measurement from a different day. The highest unpaired t test value for each data set is shown, **=P<0.001. FIG. 2B is a heat map depicting relative conjugation efficiency of isolated PGN microparticles to sfGFP. Relative conjugation efficiency was calculated by dividing the MFI of modified PGN microparticles by the MFI of D-ala-PGN microparticles. Top panel shows unnatural amino acid incorporated into PGN microparticles; left panel shows bacteria tested; bottom panel shows modified sfGFP used; parenthetical number, molar equivalents are used to non-specifically modify sfGFP. Relative conjugation efficiency is represented by scores, e.g., the higher the score, the higher the efficiency.

FIGS. 3A-3F depict characterization and immunogenicity of sfGFP-modified PGN microparticles according to aspects of this disclosure. FIG. 3A shows DLS traces of purified PGN microparticles from azaDala modified S. aureus pre- and post-conjugation with DBCO-modified-sfGFP. Pre-conjugation is shown as circles. Post-conjugation is shown as squares. FIG. 3B shows a vaccine schedule for the guinea pig immunizations. Immunizations are shown below the line and bleeds are shown above the line, all values are shown in days. FIG. 3C shows binding to sfGFP-coated ELISA plates (quantified as net absorbance at 450 nm) for serum isolated at 4 weeks post prime. FIG. 3D shows binding to sfGFP-coated ELISA plates (quantified as net absorbance at 450 nm) for serum isolated at 2 weeks post first boost. FIG. 3E shows binding to sfGFP-coated ELISA plates (quantified as net absorbance at 450 nm) for serum isolated at 2 weeks post second boost. PEP represents sfGFP-SA-Aza-Pep. KLH without adjuvant is shown as circles, KLH with Freund's adjuvant system is shown as squares, and PEP is shown as triangles. FIG. 3F illustrates biolayer interferometry (BLI) binding data of serum isolated from guinea pigs 2 weeks post second boost to sfGFP coated biosensor tips. KLH-sfGFP with Freund's adjuvant system (KLH GFP Freund's) is shown as light-shaded lines on top. KLH-sfGFP without Freund's adjuvant system (KLH-GFP) is shown as medium-shaded lines on bottom. sfGFP-SA-Aza-Pep (PEP) is shown as a dark-shaded line in the middle.

FIGS. 4A-4E demonstrates that the strain of S. aureus plays a role in overall immune response to PGN microparticles. FIG. 4A shows MFI of DBCO sfGFP-conjugated azaDala S. aureus strains (shaded bar on left). FIG. 4B shows RAW-Blue macrophage stimulation assay data comparing sfGFP-modified PGN microparticles from different strains of S. aureus. KLH with an sfGFP conjugate is shown as dark-shaded bar on left. Conjugated S. aureus strains are shown in the middle. Positive controls and negative controls are shown as unfilled bars and striped-bars, respectively, on right. Conditions are as defined in FIG. 2A. FIG. 4C shows a vaccine schedule for the mouse immunizations. Immunizations are shown below the line and bleeds are shown above the line, all values are shown in days. FIG. 4D shows total IgG EC₅₀ from ELISA binding titers of mice immunized with sfGFP-conjugated KLH or PGN microparticles from a variety of S. aureus strains. FIG. 4E shows ELISA binding titers of specific IgG subtypes from mice immunized with sfGFP-conjugated KLH or PGN microparticles from a variety of S. aureus strains.

FIGS. 5A-5F depict that PGN microparticles conjugated with SARS-CoV-2 RBD elicit a neutralizing antibody response. FIG. 5A is a schematic illustration of SARS-CoV-2 RBD (as shown as circles) conjugated to a PGN microparticle. FIG. 5B shows a vaccination schedule for the mouse immunizations. Immunizations are shown below the line and bleeds are shown above the line, all values are shown in days. FIG. 5C shows ELISA binding of serum from mice immunized with SARS-CoV-2-RBD-conjugates. FIG. 5D shows EC₅₀ derived from the curves in FIG. 5C (mean and SEM; PEP-RBD is SARS-CoV-2 RBD conjugated to SH1000 PGN). FIG. 5E shows neutralization curves of heat-inactivated serum from mice immunized with SARS-CoV-2-RBD-conjugates. FIG. 5F shows IC₅₀ derived from the curves in FIG. 5E (mean and SEM). FIGS. 5C-5F: PGN conjugated RBD (PEP-RBD) is shown as circles. KLH conjugated RBD (KLH-RBD) is shown as squares. Un-conjugated RBD is shown as triangles.

FIGS. 6A-6B depict simplified embodiments of the present disclosure. As shown, isolated PGN sacculi conjugated to a modified amino acid can be conjugated to an immunogenic peptide through click chemistry. FIG. 6B depicts the presence of a linker, for example, a PEG, that can be added to the composition. The skilled artisan would readily understand that the depiction of the embodiments of compositions as shown in FIGS. 6A-6B are not intended to be limited to a single amino acid and immunogenic peptide conjugated to the bacterial sacculus.

FIG. 7 is an embodiment of a method 100 of making an isolated PGN sacculus-immunogenic peptide composition according to the present disclosure. As shown in FIG. 7, first, a PGN sacculus (or sacculi) are isolated 101. The isolated PGN sacculus/sacculi are then chemically modified 103. In certain aspects, the chemical modification comprises incubation of the isolated sacculi with an entity to be conjugated, for example, a modified amino acid. After the chemical modification 103, an immunogenic polypeptide can be conjugated to the chemically-modified isolated PGN sacculus 105.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. TERMINOLOGY

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e. at least one) of the grammatical object of the article. By way of example, “an element” means at least one element and can include more than one element. Thus, for example, reference to “an alkyne-modified D-amino acid” includes a plurality of such amino acids and reference to “the azide-modified D-amino acid” includes reference to one or more azide-modified D-amino acids and equivalents thereof known to those skilled in the art, and so forth.

“About” is used to provide flexibility to a numerical range endpoint by providing that a given value may be “slightly above” or “slightly below” the endpoint without affecting the desired result. For example, the term “about” would indicate a range surrounding that explicit value. If “X” were the value, “about X” would indicate a value from ±1% to ±10%, preferably a value from ±1% to ±5%, and more preferably, a value from ±1% to ±3%. Thus, “about X” is to teach and provide written description support for a claim limitation of, e.g., ±100%, ±5%, or 3%.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where the phrase is interpreted in the alternative (“or”).

Recitation of ranges of values herein is merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used in this disclosure, the term “peptidoglycan” (abbreviated as “PGN”), also referred to as “murein”, is a polymer of glycosaminoglycan chains interlinked with short peptides that form the major component of the cell walls (exoskeleton) of many bacteria. The bacterial cell wall is also referred to herein as a “sacculus”. As used in this disclosure, PGN and PGN sacculus and PGN microparticle are used interchangeably. The peptidoglycan structure of both Gram-positive and Gram-negative bacteria comprises repeating disaccharide backbones of N-acetylglucosamine (NAG) and -(1-4)-N-acetylmuramic acid (NAM) that are crosslinked by peptide stem chains attached to the NAM residues. The first 2 residues of the stem peptide are generally L-alanine and D-glutamine or isoglutamine, while the last residue is typically D-alanine. In contrast, the third residue of the stem peptide is a lysine in coccoid Gram-positive bacteria (such as Staphylococcus and Streptococcus species), but a meso-diaminopimelate (mDAP) residue in both Gram-negative bacteria and many rod-shaped Gram-positive bacteria such as Listeria and Bacillus species. In the latter (DAP-type) PGN, mDAP residues from 2 adjacent stem peptides typically link directly to each other. Conversely, the stem peptides of Lys-type PGN are bridged by a variable peptide usually comprised of 2-5 glycine and serine residues. These muropeptides can be produced or modified by the activity of bacterial glycolytic and peptidolytic enzymes referred to as PGN hydrolases and autolysins.

As used herein, the terms “protein,” “peptide,” and “polypeptide” are used interchangeably herein to refer to a polymer of amino acid residues. All three terms apply to naturally occurring amino acid polymers and non-natural amino acid polymers, as well as to amino acid polymers in which one (or more) amino acid residue is an artificial chemical mimetic of a corresponding naturally occurring amino acid. As used herein, the terms encompass amino acid chains of any length, including full-length proteins, wherein the amino acid residues are linked by covalent peptide bonds.

The terms “fusion protein,” “fusion polypeptide,” and the related terms as used herein refer to polypeptide molecules, including artificial or engineered polypeptide molecules, that include two or more amino acid sequences previously found in separate polypeptide molecule, that are joined or linked in a fusion protein amino acid sequence to form a single polypeptide or joined via a chemical linker. For example, a fusion protein can be an engineered recombinant protein containing amino acid sequence from at least two unrelated proteins that have been joined together, via a peptide bond, to make a single protein. In this context, proteins are considered unrelated, if their amino acid sequences are not normally found joined together via a peptide bond in their natural environment, for example, inside a cell. The amino acid sequences of a fusion protein are encoded by corresponding nucleic acid sequences that are joined “in frame,” so that they are transcribed and translated to produce a single polypeptide. The amino acid sequences of a fusion protein can be contiguous or separated by one or more spacer, linker or hinge sequences. Fusion proteins can include additional amino acid sequences, such as, for example, signal sequences, tag sequences, and/or linker sequences.

A “domain” of a protein or a polypeptide refers to a region of the protein or polypeptide defined by structural and/or functional properties. Exemplary function properties include enzymatic activity and/or the ability to bind to or be bound by another protein or non-protein entity. For example, coronavirus Spike protein contains S1 and S2 domains.

The term “amino acid” refers to any monomeric unit that can be incorporated into a peptide, polypeptide, or protein. Amino acids include naturally-occurring α-amino acids and their stereoisomers, as well as unnatural (non-naturally occurring or “synthetic”) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate, and 0-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and their combinations. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and their combinations.

Unnatural (non-naturally occurring or “synthetic”) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to by either the commonly known three letter symbols or by the one-letter symbols recommended by the IUPAC-IUB Biochemical Nomenclature Commission.

The terms “identity,” “substantial identity,” “similarity,” “substantial similarity,” “homology” and the related terms and expressions used in the context of describing amino acid sequences refer to a sequence that has at least 60% sequence identity to a reference sequence. Examples include at least: 60%, 65%, 70%, 75%, 80%, 85%, 86%, 87%, 88%, 89%, 90%, 91%, 92%, 93%, 94%, 95%, 96%, 97%, 98%, or 99%, sequence identity, as compared to a reference sequence using the programs for comparison of amino acid sequences, such as BLAST using standard parameters. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default (standard) program parameters can be used, or alternative parameters can be designated. The sequence comparison algorithm then calculates the percent sequence identities for the test sequences relative to the reference sequence, based on the program parameters. A “comparison window” includes reference to a segment of any one of the number of contiguous positions (from 20 to 600, usually about 50 to about 200, more commonly about 100 to about 150), in which a sequence may be compared to a reference sequence of the same number of contiguous positions after the two sequences are optimally aligned. Methods of alignment of sequences for comparison are well-known. Optimal alignment of sequences for comparison may be conducted, for example, by the local homology algorithm of Smith and Waterman, 1981, by the homology alignment algorithm of Needleman and Wunsch, 1970, by the search for similarity method of Pearson and Lipman, 1988, by computerized implementations of these algorithms (for example, BLAST), or by manual alignment and visual inspection.

Algorithms that are suitable for determining percent sequence identity and sequence similarity include BLAST and BLAST 2.0 algorithms, which are described, for example, at least in Altschul et al., 1990, and Altschul et al., 1977, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (Henikoff and Henikoff, 1989). The BLAST algorithm also performs a statistical analysis of the similarity between two sequences (Karlin and Altschul, 1993).

As used herein, the terms “separating,” “isolating,” and “recovering” refer to the process of removing at least a portion (i.e., a fraction) of a first substance from a mixture containing the first substance, a second substance, and other optional substances. Separation can be conducted such that the separated substance is substantially free of at least one of the other substances present in the original mixture. For example, when the separated first substance is substantially free of the second substance, it is meant that at least about 50% (e.g., 55, 60, 65, 70, 75, 80, 85, 90, 95, or 99% (w/w)) of the second substance is removed from the isolated first substance. A “separated protein fraction” refers to a mixture containing a protein and optional excipients (e.g., buffers, detergents, and the like), wherein the protein molecules in the fraction are substantially identical. By “substantially identical,” is it meant that at least about 50% of the protein molecules (e.g., 55, 60, 65, 70, 75, 80, 85, 90, 95, or 99% (w/w)) have the same polypeptide sequence and the same level of modification with a handle moiety or other conjugate group.

As used herein, the term “immunize” refers to rendering a subject protected from an infectious disease, such as by vaccination. In the context of this disclosure, the term also refers to injection of a molecule (i.e. an immunogen) into a subject (e.g., an animal) with the purpose of producing an immune response (e.g., antibodies) against the injected molecule.

As used herein, a “protective immune response” refers to an immune response induced after administration of a vaccine composition to a subject where, upon exposure to the source of the antigenic component of the vaccine (e.g., pathogen or cell expressing the antigen), the clinical symptomatology elicited by the source are diminished.

The term “immunogenic” and the related terms, when used in the context of the present disclosure, refers to the ability of an antigen (which can be a protein, a polypeptide, or a region of a protein or a polypeptide, for example) to elicit in a subject an immune response to the specific antigen. In the context of the present disclosure, an immune response is the development in a subject of a humoral and/or a cellular immune response to an antigen. A “humoral immune response” refers to an immune response mediated by antibody molecules, including secretory (IgA) or IgG molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A cellular immune response also refers to the production of cytokines, chemokines and other such molecules produced by activated T-cells and/or other white blood cells, including those derived from CD4⁺ and CD8⁺ T-cells. Thus, an immunogenic composition can stimulate CTLs, and/or the production or activation of helper T-cells. The production of chemokines and/or cytokines may also be stimulated. An immunogenic composition may also elicit an antibody-mediated immune response. An immunogenic composition may include one or more of the following effects upon administration to a subject: production of antibodies by B-cells; and/or the activation of suppressor, cytotoxic, or helper T-cells and/or T-cells directed specifically to an antigen protein present in the immunogenic composition. Immune response elicited in the subject may serve to neutralize infectivity of a virus, such as a coronavirus, for example, SARS-CoV-2, and/or mediate antibody-complement, or antibody dependent cell cytotoxicity (ADCC) to provide protection against viral infection to an immunized subject. In some embodiments, the composition disclosed herein is an immunogenic composition. Various aspects of an immune response elicited by immunogenic compositions can be determined using standard assays, some of which are described in the present disclosure.

The term “antibody” and the related terms refer to an immunoglobulin or its fragment that binds to a particular spatial and polar organization of another molecule. Immunoglobulins include various classes and isotypes, such as IgA, IgD, IgE, IgG1, IgG2a, IgG2b and IgG3, IgG4, IgM, etc. An antibody can be monoclonal or recombinant, and can be prepared by laboratory techniques, such as by preparing continuous hybrid cell lines and collecting the secreted protein, or by cloning and expressing nucleotide sequences or their mutagenized versions coding at least for the amino acid sequences required for binding. Antibodies as referenced herein may have sequences derived from non-human antibodies, human sequence, chimeric sequences, and wholly synthetic sequences. The term “antibody” encompasses natural, artificially modified, and artificially generated antibody forms, such as humanized, human, single-chain, chimeric, synthetic, recombinant, hybrid, mutated, grafted, and in vitro generated antibodies and their fragments. The term “antibody” also includes composite forms including but not limited to fusion proteins containing an immunoglobulin moiety. “Antibody” also refers to non-quaternary antibody structures (such as camelids and camelid derivatives) and antigen-binding fragments of antibodies, minibodies, bispecific antibodies, nanobodies (also referred to as V_{H}H fragments), and diabodies. See, for example, Siontorou C G. 2013, “Nanobodies as novel agents for disease diagnosis and therapy,” Int J Nanomedicine 8:4215-4227. Antibody fragments may include Fab, Fv, F(ab′)2, Fab′, scFv, dsFv, ds-scFv, Fd, dAb, Fc, and the like. A natural antibody digested by papain yields three fragments. two Fab fragments and one Fc fragment. The Fc fragment is dimeric and contains two CH2 and two CH3 heavy chain domains. CH3 domains interact to form a homodimer. See, for example, Yang et al., 2018, “Engineering of Fc Fragments with Optimized Physicochemical Properties Implying Improvement of Clinical Potentials for Fc-Based Therapeutics” Frontiers in Immunology 8:1860. In addition, aggregates, polymers, and conjugates of immunoglobulins or their fragments can be used where appropriate.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci.

A “neutralizing antibody” is an antibody that is capable of keeping an infectious agent, such as a virus, from infecting a cell by neutralizing or inhibiting one or more parts of the life cycle of the infectious agent. For coronaviruses, neutralizing antibodies typically specifically bind to the receptor binding domain (RBD) of the Spike protein and act to disrupt or prevent interaction of the virus spike with its receptor such that virus entry into the target cell is prevented or reduced. As such, neutralizing antibodies can act to prevent or reduce the incidence of coronavirus infection.

“Virus” is used in both the plural and singular senses. “Virion” refers to a single virus. For example, the expression “coronavirus virion” refers to a single coronavirus particle.

Coronaviruses are a group of enveloped, single-stranded, RNA viruses that cause diseases in mammals and birds. Coronavirus hosts include bats, pigs, dogs, cats, mice, rats, cows, rabbits, chickens and turkeys. In humans, coronaviruses cause mild to severe respiratory tract infections. Coronaviruses vary significantly in risk factor. Some can kill more than 30% of infected subjects. The following strains of human coronaviruses are currently known: Human coronavirus 229E (HCoV-229E); Human coronavirus OC43 (HCoV-OC43); Severe acute respiratory syndrome coronavirus (SARS-CoV or SARS-CoV-1); Human coronavirus NL63 (HCoV-NL63, New Haven coronavirus); Human coronavirus HKU1 (HCoV-HKU1), which originated from infected mice, was first discovered in January 2005 in two patients in Hong Kong; Middle East respiratory syndrome-related coronavirus (MERS-CoV), also known as novel coronavirus 2012 and HCoV-EMC; and Severe acute respiratory syndrome coronavirus 2 (SARS-CoV-2), also known as 2019-nCoV or “novel coronavirus 2019.” Several variants of SARS-CoV-2 have been identified, including B.1.1.7, also known as the “UK variant,” initially detected in the United Kingdom, and B.1.351, also known as the “South Africa variant,” initially detected in South Africa in December 2020. The coronaviruses HCoV-229E, -NL63, -OC43, and -HKU1 continually circulate in the human population and cause respiratory infections in adults and children world-wide.

Spike protein (or “S protein”) is a coronavirus surface protein that is able to mediate receptor binding and membrane fusion between a coronavirus virion and its host cell. Characteristic spikes on the surface of coronavirus virions are formed by ectodomains of homotrimers of Spike protein. In comparison to trimeric glycoproteins found on other human-pathogenic enveloped RNA viruses, coronavirus Spike protein is considerably larger, and totals nearly 450 kDa per trimer. Ectodomains of coronavirus Spike proteins contain an N-terminal domain named S1, which is responsible for binding of receptors on the host cell surface, and a C-terminal S2 domain responsible for fusion. S1 domain of SARS-CoV-2 Spike protein is able to bind to Angiotensin-converting enzyme 2 (ACE2) of host cells. The region of SARS-CoV-2 Spike protein S1 domain that recognizes ACE2 is a 25 kDa domain called the receptor binding domain (RBD) (Walls et al., 2020, “Structure, Function, and antigenicity of the SARS-CoV-2 Spike Glycoprotein,” Cell 181(2):281-292.e6). Analysis of sera from COVID-19 patients demonstrates that antibodies are elicited against the Spike protein and can inhibit viral entry into the host cell (Brouwer et al., 2020, “Potent neutralizing antibodies from COVID-19 patients define multiple targets of vulnerability,” Science, 369(6504):643-650). The first Cryo-EM structure of SARS-CoV-2 Spike protein is described in Wrapp et al., 2020, “Cryo-EM structure of the 2019-nCoV spike in the prefusion conformation,” Science 367 (6483):1260-1263.

The term “bioconjugation” or “bioconjugate” refers to a chemical strategy of linking one molecule to another by chemical or biological means. The resulting complexes will typically be formed from at least one biomolecule, though they can also be purely synthetic molecules with a biological application. Typically, bioconjugation involves the use of a handle moiety. For example, bioconjugation described in the present disclosure refers to forming a stable covalent link between an immunogenic polypeptide (e.g., a full-length SARS-CoV-2 protein, SAR2-CoV-2 RBD, or a fragment thereof) and a peptide-reactive handle (e.g., a maleimide moiety or a N-hydroxysuccinimide moiety).

As used herein, the term “reactive conjugation moiety” refers to a functional group in a first molecule that can be covalently bonded to a complementary functional group in a second molecule. Unless otherwise specified, a reactive conjugation moiety is a prosthetic group that is chemically appended to a polypeptide; the reactive conjugation moiety is not naturally expressed as part of the primary amino acid sequence (e.g., as an amino acid sidechain) or as a post-translational modification (e.g., a glycan). The term “complementary functional group” refers to a functional group which is capable of covalently bonding to the reactive conjugation moiety. Unless otherwise specified, the complementary functional group can be a chemically-appended prosthetic group or naturally-expressed biological functional group (e.g., a primary amine group of a lysine sidechain or a thiol group of a cysteine sidechain).

As used herein, the term “click reaction” refers to a chemical reaction characterized by a large thermodynamic driving force that usually results in irreversible covalent bond formation. Click reactions can often be conducted under aqueous conditions (e.g., physiological conditions) without producing cytotoxic byproducts. Without intending to be limiting, examples of click reactions include [3+2]cycloadditions, such as the Huisgen 1,3-dipolar cycloaddition reaction of an azide and an alkyne; thiol-ene reactions, such as the Michael addition of a thiol to a maleimide or other unsaturated acceptor; [4+1]cycloaddition reactions between an isonitrile and a tetrazine; the Staudinger ligation between an azide and an ester-functionalized phosphine or an alkanethiol-functionalized phosphine; Diels-Alder reactions (e.g., between a furan and a maleimide); and inverse electron demand Diels-Alder reactions (e.g., between a tetrazine and a dienophile such as a strained transcyclooctene).

As used herein, the term “alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. Unless otherwise specified, “substituted alkyl” groups can be substituted with one or more groups selected from halo, hydroxy, amino, alkylamino, amido, acyl, nitro, cyano, and alkoxy.

As used herein, the term “alkoxy,” by itself or as part of another substituent, refers to a group having the formula —OR, wherein R is alkyl as described above.

As used herein, the term “cycloalkyl,” by itself or as part of another substituent, refers to a saturated or partially unsaturated, monocyclic, fused bicyclic, or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene, and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈cycloalkyl, exemplary groups include, but are not limited to cyclopropyl, cyclobutyl, cyclopentyl, cyclohexyl, cycloheptyl, and cyclooctyl. When cycloalkyl is a saturated monocyclic C₃₋₆ cycloalkyl, exemplary groups include, but are not limited to, cyclopropyl, cyclobutyl, cyclopentyl, and cyclohexyl. Cycloalkyl groups can be substituted or unsubstituted. Unless otherwise specified, “substituted cycloalkyl” groups can be substituted with one or more groups selected from, for example, halo, hydroxy, amino, alkylamino, amido, acyl, nitro, cyano, and alkoxy.

As used herein, the term “alkylene” refers to an alkyl group, as defined above, linking at least two other groups (i.e., a divalent alkyl radical). The two moieties linked to the alkylene group can be linked to the same carbon atom or different carbon atoms of the alkylene group.

As used herein, the terms “halo” and “halogen,” by themselves or as part of another substituent, refer to, for example, a fluorine, chlorine, bromine, or iodine atom.

As used herein, the term “aryl,” by itself or as part of another substituent, refers to an aromatic ring system having any suitable number of carbon ring atoms and any suitable number of rings. Aryl groups can include any suitable number of carbon ring atoms, such as C₆, C₇, C₈, C₉, C₁₀, C₁₁, C₁₂, C₁₃, C₁₄, C₁₅ or C₁₆, as well as C₆₋₁₀, C₆₋₁₂, or C₆₋₁₄. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl, or biphenyl. Other aryl groups have from 6 to 10 ring members, such as phenyl or naphthyl. Some other aryl groups have 6 ring members, such as phenyl. Aryl groups can be substituted or unsubstituted. Unless otherwise specified, “substituted aryl” groups can be substituted with one or more groups selected from halo, hydroxy, amino, alkylamino, amido, acyl, nitro, cyano, and alkoxy.

As used herein, the term “heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as, for example, N, O, or S. Additional heteroatoms can also be useful, including, but not limited to, B, Al, Si and P. The heteroatoms can be oxidized to form moieties such as, but not limited to, —S(O)— and —S(O)₂—, Heteroaryl groups can include any number of ring atoms, such as C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of heteroatoms can be included in the heteroaryl groups, such as 1, 2, 3, 4; or 5, or 1 to 2, 1 to 3, 1 to 4, 1 to 5, 2 to 3, 2 to 4, 2 to 5, 3 to 4, or 3 to 5. For example, heteroaryl groups can be C₅₋₈ heteroaryl, wherein 1 to 4 carbon ring atoms are replaced with heteroatoms; or C₅₋₈ heteroaryl, wherein 1 to 3 carbon ring atoms are replaced with heteroatoms; or C₅₋₆ heteroaryl, wherein 1 to 4 carbon ring atoms are replaced with heteroatoms; or C₅₋₆ heteroaryl, wherein 1 to 3 carbon ring atoms are replaced with heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. Unless otherwise specified, “substituted heteroaryl” groups can be substituted with one or more groups selected from halo, hydroxy, amino, alkylamino, amido, acyl, nitro, cyano, and alkoxy.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as, for example, pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include, for example, those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those, for example, having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those, for example, having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

As used herein, the term “heterocyclyl,” by itself or as part of another substituent, refers to a saturated ring system having from 3 to 12 ring members and from 1 to 4 heteroatoms of N, O, and S. Additional heteroatoms can also be useful, including, but not limited to, B, Al, Si and P. The heteroatoms can be oxidized to form moieties such as, but not limited to, —S(O)— and —S(O)₂—. Heterocyclyl groups can include any number of ring atoms, such as, C₃₋₆, C₄₋₆, C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of carbon ring atoms can be replaced with heteroatoms in the heterocyclyl groups, such as 1, 2, 3, or 4, or 1 to 2, 1 to 3, 1 to 4, 2 to 3, 2 to 4, or 3 to 4. The heterocyclyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocyclyl groups can also be fused to aromatic or non-aromatic ring systems to form members including, but not limited to, indoline. Heterocyclyl groups can be unsubstituted or substituted. Unless otherwise specified, “substituted heterocyclyl” groups can be substituted with one or more groups selected from halo, hydroxy, amino, oxo, alkylamino, amido, acyl, nitro, cyano, and alkoxy.

The heterocyclyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocyclyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane, and dithiane. Heterocyclyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

As used herein, the term “amino” refers to a moiety —NR₂, wherein each R group is H or alkyl. An amino moiety can be ionized to form the corresponding ammonium cation. “Dialkylamino” refers to an amino moiety wherein each R group is alkyl.

As used herein, the term “hydroxy” refers to the moiety —OH.

As used herein, the term “cyano” refers to a carbon atom triple-bonded to a nitrogen atom (i.e., the moiety —C≡N).

As used herein, the term “carboxy” refers to the moiety —C(O)OH. A carboxy moiety can be ionized to form the corresponding carboxylate anion.

As used herein, the term “amido” refers to a moiety —NRC(O)R or —C(O)NR₂, wherein each R group is H or alkyl.

As used herein, the term “nitro” refers to the moiety —NO₂.

As used herein, the term “oxo” refers to an oxygen atom that is double-bonded to a compound (i.e., O═).

As used herein, the term “salt” refers to acid salts or base salts employed in the methods and compositions described herein. Illustrative examples of pharmaceutically acceptable salts include mineral acid salts (for example, salts of hydrochloric acid, hydrobromic acid, phosphoric acid, and the like), organic acid salts (for example, salts of acetic acid, propionic acid, glutamic acid, citric acid and the like) salts, and quaternary ammonium salts (for example, salts of methyl iodide, ethyl iodide, and the like). It is understood that the pharmaceutically acceptable salts are non-toxic. Additional information on suitable pharmaceutically acceptable salts can be found in the literature, for example, in Remington: The Science & Practice of Pharmacy, 20th ed., Lippincott Williams & Wilkins, Philadelphia, Pa., 2000, which is incorporated herein by reference.

The term “cancer” or “tumor” is intended to include any member of a class of diseases characterized by the uncontrolled growth of aberrant cells. The term includes all known cancers and neoplastic conditions, whether characterized as malignant, benign, recurrent, soft tissue, or solid, and cancers of all stages and grades including advanced, pre- and post-metastatic cancers. Examples of different types of cancer include, but are not limited to, gynecological cancers (e.g., ovarian, cervical, uterine, vaginal, and vulvar cancers); lung cancers (e.g., non-small cell lung cancer, small cell lung cancer, mesothelioma, carcinoid tumors, lung adenocarcinoma); breast cancers (e.g., triple-negative breast cancer, ductal carcinoma in situ, invasive ductal carcinoma, tubular carcinoma, medullary carcinoma, mucinous carcinoma, papillary carcinoma, cribriform carcinoma, invasive lobular carcinoma, inflammatory breast cancer, lobular carcinoma in situ, Paget's disease, Phyllodes tumors); digestive and gastrointestinal cancers such as gastric cancer (e.g., stomach cancer), colorectal cancer, gastrointestinal stromal tumors (GIST), gastrointestinal carcinoid tumors, colon cancer, rectal cancer, anal cancer, bile duct cancer, small intestine cancer, and esophageal cancer; thyroid cancer; gallbladder cancer; liver cancer; pancreatic cancer; appendix cancer; prostate cancer (e.g., prostate adenocarcinoma); renal cancer (e.g., renal cell carcinoma); cancer of the central nervous system (e.g., glioblastoma, neuroblastoma, medulloblastoma); skin cancer (e.g., melanoma); bone and soft tissue sarcomas (e.g., Ewing's sarcoma); lymphomas; choriocarcinomas; urinary cancers (e.g., urothelial bladder cancer); head and neck cancers; and bone marrow and blood cancers (e.g., acute leukemia, chronic leukemia (e.g., chronic lymphocytic leukemia), lymphoma, multiple myeloma). As used herein, a “tumor” comprises one or more cancerous cells.

### II. INTRODUCTION

The present disclosure provides modified Staphylococcus aureus peptidoglycan saccului (PGN) compositions comprising at least one modified D-amino acid, and having an immunogenic polypeptide attached thereto via a chemical linker, e.g., a triazole moiety. Compositions as described herein are unique, biodegradable, and capable of eliciting an immune response, making them ideal carriers for subunit vaccines. The disclosed compositions are easily adaptable and can be produced at scale. Additionally, this disclosure provides for prophylactic and therapeutic methods using the provided compositions.

The inventors designed, generated, characterized and demonstrated the efficacy of compositions as described herein comprising whole, isolated bacterial peptidoglycan from S. aureus as a novel microparticle vaccine scaffold. The PGN microparticles contain bio-orthogonal chemical reaction handles allowing for site-specific attachment of immunogenic proteins. The inventors determined that S. aureus PGN microparticles with azido-D-alanine incorporated therein yield robust conjugation to antigens. The inventors demonstrated efficacy of the provided compositions, for example, as a vaccine in a guinea pig immunization model, finding it comparable to the conventional carrier protein KLH. The inventors further demonstrated efficacy of the provided compositions comprising a SARS-CoV-2 receptor binding domain (RBD) in mice, eliciting the production of neutralizing antibody titers comparable to that elicited by KLH-conjugated RBD. These findings demonstrate that the provided compositions comprising chemically-modified S. aureus PGN sacculi can serve as a conjugatable and biodegradable microparticle scaffold that allow for facile vaccine production and elicits a robust immune response toward an immunogenic polypeptides.

PGN sacculi can be produced in large scale from liters of bacterial cultures and provide an opportunity for a scalable vaccine microparticle, as showcased here. PGN is naturally degraded by serum lysozymes and is regularly cleared from the body without significant systemic inflammation. PGN can also stimulate the immune system through, for example, TLR2 and NOD1 and NOD2 receptors. As described herein, PGN sacculi can be modified with synthetic D-amino acid residues bearing chemical handles by harnessing the promiscuity of the bacterium's biosynthetic cellular machinery. These chemical handles have been used to covalently attach a variety of molecules to the surface of the PGN sacculi. An advantage of conjugating the immunogenic polypeptide to the PGN sacculi by covalent bonds is co-delivery of these components to the same subset of immune cells in order to trigger the desired immune responses.

### III. COMPOSITIONS

The compositions provided herein can comprise a modified bacterial peptidoglycan (PGN) sacculus comprising at least one azide-modified D-amino acid and having an immunogenic polypeptide attached thereto via a chemical moiety, particularly, e.g., a triazole moiety.

### A. Modified Bacterial Peptidoglycan Sacculus

In some embodiments, the modified PGN sacculus is a peptidoglycan sacculus isolated from Staphylococcus aureus. In some embodiments, the modified PGN sacculus is a S. aureus strain. For example, the PGN sacculus can be from a S. aureus subspecies aureus Rosenbach strain (such as ATCC 25923 or ATCC 29213), or other S. aureus strains (such as SH1000, RN4220, or RN4220 (ΔTarO)). In some embodiments, the modified PGN is a S. aureus peptidoglycan sacculus isolated from S. aureus strain SH1000. Additional exemplary S. aureus strains are also described in the literature, for example, in Renz, A. and Dräger, A., npj Syst Biol Appl. Vol. 7, Art. No. 30 (2021); doi.org/10.1038/s41540-021-00188-4 and Spaulding et al., Front. Cell. Infect. Microbiol. Vol. 2, Art. 18 (2012); PMID: 2291%19. In some instances, the PGN sacculus is an entire sacculus. In some instances, the PGN scculus comprises digested muropeptides.

Modifications or variations to the basic PGN structure occur frequently amongst bacterial species. Many modifications are species specific, due to the expression of unique synthetic, modifying, or degradative enzymes. Substitutions and modifications to the basic PGN structure occur in both the peptide stem and bridge regions and in the disaccharide backbone. In some instances, the disaccharide backbone can be modified by addition of glycolic acid, for example, via N-acylation, to muramic acid residues. It is also common for bacteria to deacetylate N-acetyl glucosamine residues. For example, Staphylococcus aureus employs O-acetylation as an alternative method for modification of PGN sugars.

The modified PGN sacculus provided herein can comprise an unnatural amino acid residue derivative, for example a modified D-amino acid residue, that includes a bioorthogonal functional group (e.g., a reactive conjugation moiety) such as: an azide, an alkyne, a phosphine, a thiol, a maleimide, a N-hydroxysuccinimide, or an isonitrile. In some embodiments, the modified D-amino acid residue is an azide-modified D-amino acid residue, an azido-dipeptide, an azido-tripeptide, an azido-tetrapeptide, or other short-chain azido peptide. In some embodiments, the modified D-amino acid residue is azido-D-alanine. In some embodiments, the modified PGN comprises a plurality of modified D-amino acid residues. The modified PGN can include a plurality of the same modified D-amino acid residues or a plurality of different modified D-amino acid residues, the different modified D-amino acid residues including the same or different bioorthogonal functional groups. Reactive conjugation moieties that can be incorporated into the modified PGN via the modified D-amino acid residues are described in Section C herein. Incorporation of modified D-amino acid residues into PGN sacculus is generally performed by growing the bacteria in an appropriate bacteria culture medium containing the desired modified D-amino acid residue(s) to be incorporated, although other methodology may be used. Such methodology is described in the Examples herein as well as, for example, in U.S. Pat. Nos. 9,303,068 and 9,789,180, which are incorporated herein in their entireties for all purposes.

Given the size of PGN sacculi and the relative ease of incorporating distinct clickable handles on the biorthogonal functional group therein (simply growing the cells in broth containing an additional D-amino acids), it is possible to produce compositions simultaneously containing the immunogenic polypeptide as well as a multiplicity of additional components, such as TLR agonists, T-cell epitopes, or cancer neo-antigens as described herein and/or adjuvants as described herein, conjugated thereto.

### B. Antigens and Immunogenic Polypeptides

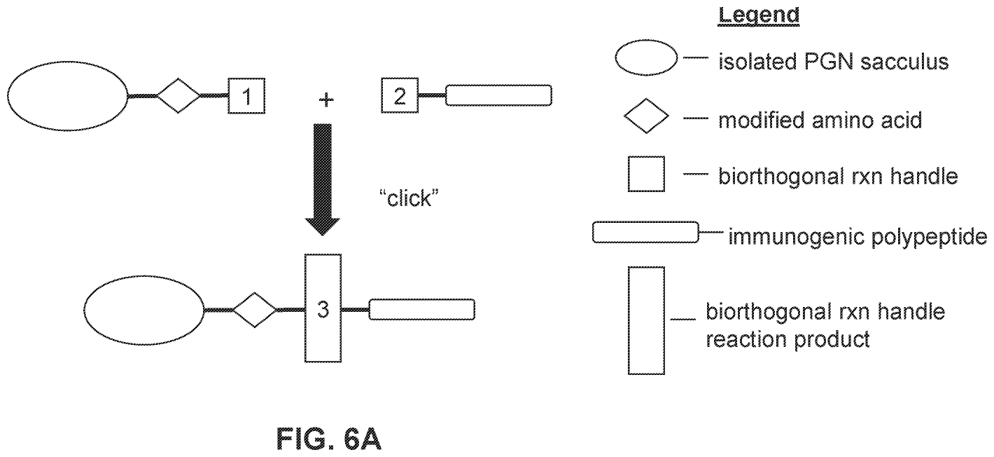

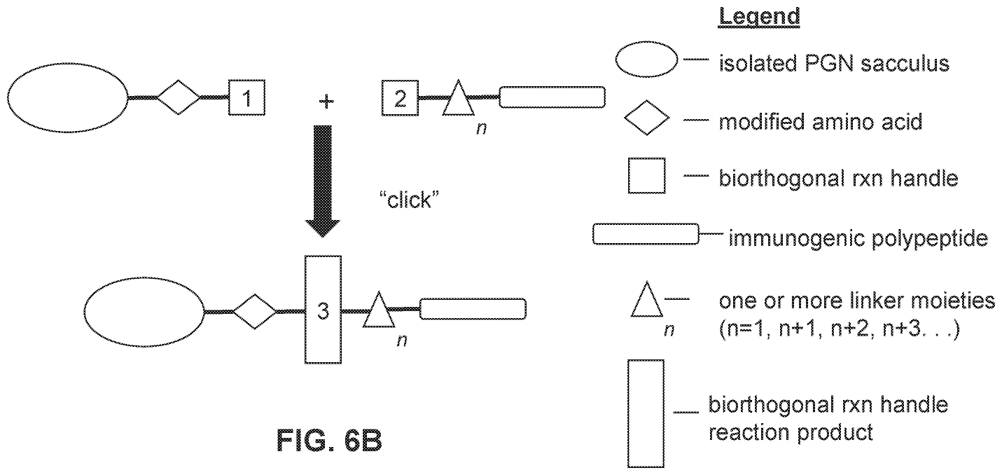

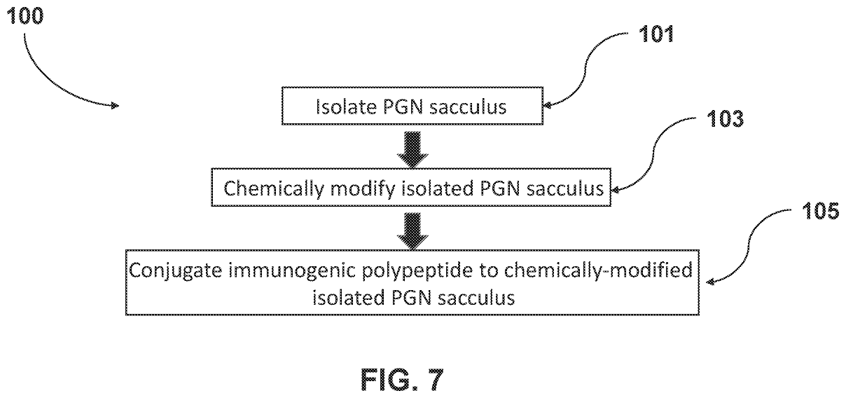

As described herein, the composition comprises a bacterial PGN sacculus and an immunogenic polypeptide chemically conjugated thereto (see, for example, FIGS. 6A-6B, and embodiment of method 100 in FIG. 7). Immunogenic polypeptides are capable of eliciting in a subject an immune response to a specific antigen (e.g., the polypeptides). In the context of the present disclosure, an immune response is the development in a subject of a humoral and/or a cellular immune response to an antigen. In some embodiments, the composition is formulated as a subunit vaccine against a viral infection and/or a cancer. In some embodiments, the composition comprises a plurality of antigens conjugated thereto, the plurality of antigens comprising at least one immunogenic polypeptide. The number of antigens (e.g., immunogenic polypeptides) attached to the bacterial PGN sacculus can range from 1 to 20 or more.

In some embodiments, the composition comprises an antigen (e.g., an immunogenic polypeptide) that is functionalized with a reactive conjugation moiety using a crosslinker reagent as described below in Section C. In some embodiments, the composition can further comprise an adjuvant as described below in Section IV attached to the PGN sacculus or combined therewith.

Any antigen from any disease, disorder, or condition may be used in (or otherwise in conjunction with) compositions as provided herein, as the skilled artisan would readily understand. Exemplary antigens include but are not limited to bacterial antigens, viral antigens, parasitic antigens, allergens, autoantigens and tumor-associated antigens.

Immunogenic polypeptides can be natural, synthetic, semi-synthetic, or naturally-occurring inorganic or organic molecules. For instance, immunogenic polypeptides in the context of the present disclosure can be isolated bacterial, viral, fungal, plant and/or animal molecules or recombinant molecules. As an example, the immunogenic polypeptides can be bacterial antigens (e.g., Staphylococcus antigens), viral antigens (e.g., SARS-CoV-2), fungal antigens (e.g., Aspergillus antigens), plant antigens (e.g., haptens), and/or animal antigens (e.g., human leukocyte antigen (HLA), cancer neoantigens or proteins expressed in cancer cells). Useful immunogenic polypeptides can also include variants of bacterial polypeptides, viral polypeptides, parasitic polypeptides, allergen polypeptides, autoantigens, and tumor-associated antigens. In some instances, the immunogenic polypeptide can be a fusion protein of an antigen and another protein or protein domain, such as, for example, another immunogenic polypeptide or an adjuvant.

In some embodiments, the antigen comprises a polysaccharide antigen. The polysaccharide antigen can be, for example, a bacterial polysaccharide (e.g., a Mycoobacterium polysaccharide such as a Mycoobacterium tuberculosis polysaccharide, a Pneumococcal polysaccharide such as a Streptococcus pneumoniae polysaccharide) or a tumor-associated carbohydrate antigen. Such polysaccharides can be functionalized with complementary functional groups using linking reagents as described above, prior to reaction with bacterial PGNs having reactive conjugation moieties.

### 1. Viral Proteins

Antigens that can be employed include, but are not limited to, antigens associated with infectious agents such as: hepatitis A virus, hepatitis B virus, hepatitis C virus, hepatitis D virus, hepatitis E virus, human influenza A virus (e.g., H1N1, H3N2, H2N2, H5N1, N7N9, or N9N2 influenza), human influenza B virus, dengue virus, Ebola virus, West Nile virus, Zika, virus, vaccinia virus, variola virus, human immunodeficiency virus (HIV), respiratory syncytial virus, herpes simplex virus 1, herpes simplex virus 2, human papillomavirus, Listeria spp. (e.g., L. monocytogenes), Clostridium spp. (e.g., C. difficile, C. perfringens, C. chauvoei, C. septicum, C. noiyi, and C. sordellii), Mycobacterium spp. (e.g., M. tuberculosis complex species and M. avium complex species), Francisella spp. (e.g., F. tularensis and F. novicida), Yersinia pestis (plague), and malarial pathogens (e.g., P. falciparum and P. vivax).

In some embodiments, the immunogenic polypeptide can be a viral protein. Examples of viral proteins include, but are not limited to: influenza hemagglutinin (monomer or trimer), influenza neuraminidase (monomer or tetramer), influenza matrix-1 protein (M1; monomer or tetramer), influenza matrix-2 protein (M2; monomer or tetramer), influenza nucleoprotein (NP), dengue virus envelope protein (monomer or multimer), dengue virus nonstructural protein 1 (NS1), and HIV envelope protein (monomer or trimer), or a fragment of any thereof. Examples of bacterial antigens include, but are not limited to: M. tuberculosis Ag85A and malarial pathogen proteins such as Plasmodium sp. circumsporozoite protein (CSP), P. falciparum PF3D7_1136200, P. falciparum PF3D7_0606800, P. falciparum merozoite surface proteins (e.g., MSP2, MSP3, MSP11), P. falciparum RhopH3, P. falciparum P41, P. vivax P41, Plasmodium sp. apical membrane antigen 1 (AMA1), P. falciparum Pfl 13, and P. falciparum MSP7-related proteins (e.g., MSRP1). The immunogenic polypeptide may contain a naturally-occurring protein sequence or a modified protein sequence.

In some embodiments, the immunogenic polypeptide comprises a coronavirus antigen or a fragment thereof. In some embodiments, the immunogenic polypeptide comprises a SARS-CoV antigen and/or a fragment thereof. In some embodiments, the immunogenic polypeptide comprises a SARS-CoV-2 antigen and/or a fragment thereof. In some embodiments, the immunogenic polypeptide comprises a SARS-CoV-2 Spike protein and/or a fragment thereof. In some embodiments, the immunogenic polypeptide comprises a SARS-CoV-2 Spike protein receptor binding domain (RBD) and/or a fragment thereof.

Some embodiments of the immunogenic polypeptides described in the present disclosure include a polypeptide (or a fragment thereof) of a Spike protein of a coronavirus capable of infecting humans (“human coronaviruses”), including, but not limited to, human betacoronaviruses, for example, SARS-CoV, MERS-CoV, and SARS-CoV-2. Some embodiments of the immunogenic polypeptides described in the present disclosure include a polypeptide or a fragment thereof of a Spike protein of a coronavirus capable of infecting non-human animals including, but not limited to, BatCoV RaTG13, Bat SARSr-CoV ZXC21, Bat SARSr-CoV ZC45, BatSARSr-CoV WIV1, or other coronavirus.

In some embodiments, the immunogenic polypeptide is a SARS-CoV-2 Spike protein comprising an amino acid sequence having at least 50%, at least 55%, at least 60%, at least 65%, at least 70%, at least 70%, at least 80%, at least 85%, at least 90%, at least 95%, at least 96%, at least 97%, at least 98%, at least 99%, or more identity with SEQ ID NO:1. In some embodiments, the immunogenic polypeptide is a SARS-CoV-2 Spike protein comprising an amino acid sequence having between 10%-100%, 20%-90%, 30%-80%, 40%-70%, 60%-80%, or 70%-90% (or greater) identity with SEQ ID NO:1. In some embodiments, the immunogenic polypeptide is a SARS-CoV-2 Spike protein comprising an amino acid sequence having between 10%-100%, 10%-90%, 20%-90%, 30%-90%, 40%-90%, 60%-90%, or 70%-90% (or greater) identity with SEQ ID NO:1. In some embodiments, the immunogenic polypeptide is a SARS-CoV-2 Spike protein comprising an amino acid sequence having between 10%-80%, 10%-70%, 10%-60%, 10%-50%, 10%-40%, 10%-30%, 10%-20%, 20%-60%, 30%-50%, 40%-90%, 60%-90%, or 70,%-90% (or greater) identity with SEQ ID NO: 1. In some embodiments, the immunogenic polypeptide is a SARS-CoV-2 Spike protein comprising an amino acid sequence having between 90-99%, 90%-98%, 90%-97%, 90%-96%, 90%-95%, 90%-94%, 90%-93%, 9/%-92%, or 90-91% (or greater) identity with SEQ ID NO:1. In some embodiments, the immunogenic polypeptide is a SARS-CoV-2 Spike protein comprising an amino acid sequence having between 85-99%, 85%-98%, 85%-97%, 85%-96%, 85%-95%, 85%-94%, 85%-93%, 85%-92%, 85%-91%, 85%-90%, 85-89%, 85-88%, 85-87%, or 85-86% (or greater) identity with SEQ ID NO:1. In some embodiments, the immunogenic polypeptide is a SARS-CoV-2 Spike protein comprising an amino acid sequence having about 85% identity with SEQ ID NO:1. In some embodiments, the immunogenic polypeptide is a SARS-CoV-2 Spike protein comprising an amino acid sequence having about 90% identity with SEQ ID NO:1. In some embodiments, the immunogenic polypeptide is a SARS-CoV-2 Spike protein comprising an amino acid sequence having about 95% identity with SEQ ID NO:1.

In some embodiments, the immunogenic polypeptide comprises a naturally occurring SARS-CoV and/or SARS-CoV-2 Spike protein receptor binding domain (RBD) or a truncated portion thereof. For example, a RBD can comprise amino acid residues 319-541 of the Spike protein (SEQ ID NO:2). In some embodiments, the immunogenic polypeptide comprises a variant of SARS-CoV and/or SARS-CoV-2 RBD (e.g., having at least 50%, at least 55%, at least 60%, at least 65%, at least 70%, at least 75%, at least 80%, at least 85%, at least 90%, at least 95%, at least 96%, at least 97%, at least 98%, at least 99%, or more identity with SEQ ID NO:2). In some embodiments, the immunogenic polypeptide comprises a modified or mutated SARS-CoV and/or SARS-CoV-2 RBD (SEQ ID NO:2). In some embodiments, the immunogenic polypeptide is a SARS-CoV-2 Spike protein comprising an amino acid sequence having between 10%-100%, 20%-90%, 30%-80%, 40%-70%, 60%-80%, or 70%-90% (or greater) identity with SEQ ID NO:2. In some embodiments, the immunogenic polypeptide is a SARS-CoV-2 Spike protein comprising an amino acid sequence having between 10%-100%, 10%-90%, 20%-90%, 30%-90%, 40%-90%, 60%-90%, or 70%-90% (or greater) identity with SEQ ID NO:2. In some embodiments, the immunogenic polypeptide is a SARS-CoV-2 Spike protein comprising an amino acid sequence having between 10%-80%, 10%-70%, 10%-60%, 10%-50%, 10%-40%, 10%-30%, 10%-20%, 20%-60%, 30%-50%, 40%-90%, 60%-90%, or 70%-90% (or greater) identity with SEQ ID NO:2. In some embodiments, the immunogenic polypeptide is a SARS-CoV-2 Spike protein comprising an amino acid sequence having between 90-99%, 90%-98%, 90%-97%, 90%-96%, 90%-95%, 90%-94%, 90%-93%, 90%-92%, or 90-91% (or greater) identity with SEQ ID NO:2. In some embodiments, the immunogenic polypeptide is a SARS-CoV-2 Spike protein comprising an amino acid sequence having between 85-99%, 85%-98%, 85%-97%, 85%-96%, 85%-95%, 85%-94%, 85%-93%, 85%-92%, 85%-91%, 85%-90%, 85-89%, 85-88%, 85-87%, or 85-86% (or greater) identity with SEQ ID NO:2.

Some embodiments of the immunogenic polypeptide may contain a naturally occurring (or “wild-type”) amino acid sequence of coronavirus Spike protein (SEQ ID. NO:1) or a portion thereof. Some non-limiting examples of such wild-type sequences are: a wild-type amino acid sequence of S1 domain of a coronavirus Spike protein; a wild-type amino acid sequence of a receptor binding domain (RBD) of a coronavirus Spike protein (SEQ ID NO:2); or a wild-type amino acid sequence of a coronavirus Spike protein with one or more C-terminal, N-terminal, or middle portions deleted. One example is a wild-type amino acid sequence of a coronavirus Spike protein with a C-terminal deletion encompassing the heptad repeat 2 (HR2) amino acid sequence.

Some other examples of wild-type amino acid sequences of a coronavirus Spike protein are the sequences that contain mutations, in comparison to SEQ ID NO:1, found in naturally occurring SARS-CoV-2 strains, which can also be referred to as “variants.” See, for example, PCT Application No: PCT/US2021/047885 and Table 1. One such example is a wild-type amino acid sequence of a coronavirus Spike protein having a deletion (in reference to SEQ ID NO:1) of residues 69-70 and residue 144, as found in strain SARS-CoV-2 VUI 202012/01 in SARS-CoV-2 variant lineage B.1.1.7. Another example is a wild-type amino acid sequence of a coronavirus Spike protein having a D to G substitution at residue 614, (in reference to SEQ ID NO:1), as found in SARS-CoV-2 variant D614G. Another example is a wild-type amino acid sequence of a coronavirus Spike protein having the substitutions (in reference to SEQ ID NO:1) S13I, W152C, L452R, and D614G, as found in SARS-CoV-2 variant B.1.429. Another example is a wild-type amino acid sequence of a coronavirus Spike protein having substitutions (in reference to SEQ ID NO:1) L18F, T20N, P26S, D138Y, R190S, K417T, E484K, N501Y, D614G, H655Y, T10271, as found in SARS-CoV-2 variant P1. Yet another example is a wild-type amino acid sequence of a coronavirus Spike protein having substitutions (in reference to SEQ ID NO:1) L18F, D80A, D215G, 242-244 del, R2461, K417N, E484K, N501Y, D614G, and/or A701V, as found in SARS-CoV-2 variant B.1.351. One more example is a wild-type amino acid sequence of a coronavirus Spike protein having a deletion (in reference to SEQ ID NO:1) of residues 69-70 and residue 144, and substitutions (in reference to SEQ ID NO:1) N501Y, A570D, D614G, P681H, T716I, S982A, D1118H, as found in SARS-CoV-2 variant B.1.1.7. One more example is a wild-type amino acid sequence of a coronavirus Spike protein having a deletion (in reference to SEQ ID NO:1) of residues 156-157, and substitutions (in reference to SEQ ID NO:1) T19R, G142D, R158G, L452R, T478K, D614G, P681R, and D950N, as found in SARS-CoV-2 variant B.1.617.2. An additional example is a wild-type amino acid sequence of a coronavirus Spike protein as found in SARS-CoV-2 variant B.1.1.529 having deletions (in reference to SEQ ID NO:1) of amino acid residues 69-70, 143-145, and 211 and substitutions (in reference to SEQ ID NO:1) A67V, T951, G142D, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681K N764K, D796Y, N856K, Q954H, N969K, and/or L981F. Additional examples include the sequence of other naturally occurring strains having a deletion of a few residues (e.g., 1-5) within the coronavirus Spike protein before HR2 amino acid sequence. Some of the features of the above amino acid sequences of a coronavirus Spike protein are summarized in Table 1. It is to be understood that, in some examples, various features and mutations of the wild-type amino acid sequences of a coronavirus Spike protein, including but not limited to those discussed above and summarized above, can be found in various combinations and subcombinations.

TABLE 1
Exemplary features (in reference to SEQ ID NO: 1) found
in amino acid sequences of a coronavirus Spike protein.

| SARS-CoV-2 | SARS-CoV-2 | SARS-CoV-2 | SARS-CoV-2 | SARS-CoV-2 | SARS-CoV-2 |
|---|---|---|---|---|---|
| D614G | B.1.1.7 | B.1.351 | P.1 | B.1.429 | B.1.617.2 |
| D614G | 69-70 del | L18F | L18F | S13I | T19R |
| | 144 del | D80A | T20N | W152C | G142D |
| | N501Y | D215G | P26S | L452R | 156-157 del |
| | A570D | 242-244 del | D138Y | D614G | R158G |
| | D614G | R246I | R190S | | L452R |
| | P681H | K417N | K417T | | T478K |
| | T716I | E484K | E484K | | D614G |
| | S982A | N501Y | N501Y | | P681R |
| | D1118H | D614G | D614G | | D950N |
| | | A701V | H655Y | | |
| | | | T1027I | | |

### 2. Cancer Proteins

In some embodiments, the immunogenic polypeptide comprises a cancer neoantigen and/or a protein over-expressed or abnormally expressed by a cancer cell.

Mutations occurring in tumor cells can generate novel epitopes of self-antigens, which are referred to as neoepitopes or neoantigens. (see, for example, Zheng et al., 2021. “Neoantien: A new breakthrough in tumor immunotherapy. Front Immunol. 12:672356. doi: 10.3389/fimmu.2021.672356). In general, neoantigens are tumor-specific antigens (e.g., polypeptides) that form on cancer cells when certain mutations occur in tumor cell DNA. Neoantigens are not expressed on the surface of normal cells. As new antigens, they have not previously been presented to or recognized by the immune system of the subject in which they are expressed. Neoantigens can arise through a variety of mutational events (e.g., point mutations, insertions or deletions (indels), alternative splicing, and/or gene rearrangement), with the numbers and types of mutation varying by cancer type. In tumors driven by viruses, viral proteins can be considered an alternative class of neoantigen. Tumors with a high mutational burden are likely to have several candidate neoantigens for vaccine formulation. Fewer neoantigens are likely to be present in tumors with a low mutational burden, which might restrict the number of immunogenic epitopes; however, the induction of neoantigen-specific immune responses using immunotherapy or therapeutic vaccination remains a possibility within this category of tumors.

In some embodiments, the antigen comprises a tumor-associated antigen (TAA) related to tumors or cancers as described herein. Tumor-associated antigens also include tumor-associated carbohydrate antigens (TACAs) such as Tn antigen (i.e., serine- or threonine-linked N-acetylgalactosamine), sialyl-Tn antigen (Neu5Aca2-6GalNAc), Thomsen-Friedenreich antigen (Tf; Galβ1-3GalNAcα1), gangliosides (including GD2, GD3, GM2, GM3), globosides (including Globo-H, Gb3, Cb4, and Gb5), sialyl Lewisx (Neu5Aca2-3Galβ1-4[Fucα1-3]GlcNAc), Lewis' (Galβ1-4[Fucα1-3]GlcNAc), Lewis^{y} (Fucα1-3(Fucα1-2Galβ1-4)GclNAc, and the like.

Neoantigens can be recognized by tumor-infiltrating cytotoxic CD8⁺ T cells, and increased immune cell infiltration and the related cytotoxicity signatures have been observed in tumors with a higher neoantigen load. Accordingly, neoantigen presentation and load have been positively correlated with prognosis in patients with a variety of cancers and with benefit from immune-checkpoint inhibitors (ICIs) in patients with melanoma, non-small-cell lung cancer (NSCLC) or colorectal cancer with mismatch-repair deficiency. Together, these studies highlight the potential therapeutic benefit of developing immunotherapies that specifically “train” the immune system to target neoantigens.

Vaccines predicated on neoantigens rather than traditionally used TAAs have several advantages. First, neoantigens are exclusively expressed by tumor cells and can, therefore, elicit truly tumor-specific T cell responses, thereby preventing “off-target” damage to nonmalignant tissues. Second, neoantigens are de novo epitopes derived from somatic mutations, which presents the possibility to circumvent T cell central tolerance of self-epitopes and thus induce immune responses to tumors. Personalized neoantigen-based vaccines therefore afford the opportunity to boost tumor-specific immune responses and add an additional tool to the immunotherapy toolbox. Furthermore, the potential of these vaccine-boosted neoantigen-specific T cell responses to persist and provide post-treatment immunological memory presents the possibility of long-term protection against disease recurrence.

In some instances, the immunogenic polypeptide can be a self-antigen abnormally expressed or overexpressed in tumor cells, termed tumor-associated antigens (TAAs). Early studies with TAAs were largely unsuccessful in generating clinically effective antitumor immune responses, probably owing to the TAA-specific T cells being subject to central and/or peripheral tolerance. The poor immunogenicity of TAAs can be addressed by incorporation of such antigens into the compositions provided herein.

### C. Bioconjugation

As set forth above, compositions according to the present disclosure can include: 1) a bacterial PGN comprising a first reactive conjugation moiety, and 2) an immunogenic polypeptide comprising a second reactive conjugation moiety. In some embodiments, the first and second reactive conjugation moieties have reacted so as to covalently bond the bacterial PGN to the immunogenic polypeptide. In some embodiments, the first reactive conjugation moiety is installed in the bacterial PGN for further modification via one or more click reactions. Accordingly, some embodiments disclosed herein provide bacterial PGN wherein the first reactive conjugation moiety is a click conjugation moiety, such as: an azide, an alkyne, a phosphine, a thiol, a maleimide, an N-hydroxysuccinimide, or an isonitrile. When the first reaction moiety is an azido-D-alanine residue, for example, conjugation can be conducted using an immunogenic polypeptide bearing a cyclooctyne moiety as the second reactive moiety. Alternatively, an azido-D-alanine residue may first be reacted with a cycloalkyne-containing heterobifunctional cross-linker (e.g., a cyclooctyne-maleimide crosslinker). The resulting maleimide-bearing PGN can be then reacted with a cysteine residue in the immunogenic polypeptide to form the final immunogenic composition.

Bonding of an immunogenic polypeptide to a bacterial PGN is conducted under conditions for forming covalent bonds between reactive conjugation moieties (e.g., between a click conjugation moiety on a bacterial PGN and a complementary click functional group on an immunogenic polypeptide). The reactions may be conducted at any suitable temperature. In general, the reactions are conducted at a temperature of from about 4° C. to about 50° C. The reactions can be conducted, for example, at about 25° C. to about 37° C. In certain aspects, the reactions can be conducted at about 25° C. or about 37° C. The reactions can be conducted at any suitable pH. In general, the reactions are conducted at a pH of from about 4.5 to about 10. The reactions can be conducted, for example, at a pH of from about 5 to about 9, about 6 to about 8, or about 7. In some embodiments, the reaction is conducted at a pH ranging from 7.2 to 7.5, about 7.3 to about 7.5, about 7.4 to about 7.5, about 7.2 to about 7.3, about 7.2 to about 7.4, about 7.3 to about 7.4, and the like. The reactions can be conducted for any suitable length of time. In general, the reaction mixtures are incubated under suitable conditions for anywhere between about 1 minute and a few days (up to 3 to 4 days). The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Other reaction conditions may be used, depending on the particular bacterial PGN, antigen, and/or the specific reactive conjugation moieties employed.

Reaction mixtures for forming the conjugates can contain additional reagents of the sort typically used in bioconjugation reactions. For example, in certain embodiments, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, tetrahydrofuran, acetone, and acetic acid), salts (e.g., NaCl, KCl, CaCl₂, and salts of Mn²⁺ and Mg²⁺), detergents/surfactants (e.g., a non-ionic surfactant such as N,N-bis[3-(D-gluconamido)propyl]cholamide, polyoxyethylene (20) cetyl ether, dimethyldecylphosphine oxide, branched octylphenoxy poly(ethyleneoxy)ethanol, a polyoxyethylene-polyoxypropylene block copolymer, t-octylphenoxypolyethoxyethanol, polyoxyethylene (20) sorbitan monooleate, and the like; an anionic surfactant such as sodium cholate, N-lauroylsarcosine, sodium dodecyl sulfate, and the like; a cationic surfactant such as hexdecyltrimethyl ammonium bromide, trimethyl(tetradecyl) ammonium bromide, and the like; or a zwitterionic surfactant such as an amidosulfobetaine, 3-[(3-cholamidopropyl)dimethyl-ammonio]-1-propanesulfonate, and the like), chelators (e.g., ethylene glycol-bis(2-aminoethylether)-N,N,N′,N′-tetraacetic acid (EGTA), 2-{(2-[bis(carboxymethyl)amino]ethyl}(carboxymethyl)amino)acetic acid (EDTA), and 1,2-bis(o-aminophenoxy)ethane-N,N,N′,N′-tetraacetic acid (BAPTA)), and reducing agents (e.g., dithiothreitol (DTT), β-mercaptoethanol (BME), and tris(2-carboxyethyl)phosphine (TCEP)). Buffers, cosolvents, salts, detergents/surfactants, chelators, and reducing agents can be used at any suitable concentration, which can be readily determined by one of skill in the art. In general, buffers, cosolvents, salts, detergents/surfactants, chelators, and reducing agents are included in reaction mixtures at concentrations ranging from about 1 μM to about 1 M, or any range therein. For example, a buffer, a cosolvent, a salt, a detergent/surfactant, a chelator, or a reducing agent can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M. In other examples, a buffer, a cosolvent, a salt, a detergent/surfactant, a chelator, or a reducing agent can be included in a reaction mixture at a concentration of about 1 μM to about 1000 μM, about 10 μM to about 990 μM, about 20 μM to about 980 μM, about 30 μM to about 970 μM, about 40 μM to about 960 μM, about 50 M to about 950 μM, about 60 μM to about 940 μM, about 70 μM to about 930 μM, about 80 μM to about 920 μM, about 90 M to about 910 μM, about 100 μM to about 900 μM, about 110 μM to about 890 μM, about 120 μM to about 880 μM, about 130 M to about 870 μM, about 130 μM to about 860 μM, about 140 μM to about 850 μM, about 150 μM to about 840 μM, about 160 μM to about 830 μM, about 170 μM to about 820 μM, about 180 μM to about 810 μM, about 190 μM to about 800 μM, about 200 μM to about 790 μM, about 210 μM to about 780 μM, about 220 μM to about 770 μM, about 230 μM to about 760 μM, about 240 μM to about 750 μM, about 250 μM to about 740 μM, about 260 μM to about 730 μM, about 270 μM to about 720 μM, about 280 μM to about 710 μM, about 290 μM to about 700 μM, about 300 μM to about 690 μM, about 310 μM to about 680 μM, about 320 μM to about 670 μM, about 330 μM to about 660 μM, about 340 μM to about 650 μM, about 350 μM to about 640 μM, about 360 μM to about 630 μM, about 370 μM to about 620 μM, about 380 μM to about 610 μM, about 390 μM to about 600 μM, about 400 μM to about 590 μM, about 410 μM to about 580 μM, about 420 μM to about 570 μM, about 430 μM to about 560 μM, about 440 μM to about 550 μM, about 450 μM to about 540 μM, about 460 μM to about 530 μM, about 470 μM to about 520 μM, about 480 μM to about 510 μM, about 490 μM to about 510 μM, or about 500 μM. In other examples, a buffer, a cosolvent, a salt, a detergent/surfactant, a chelator, or a reducing agent can be included in a reaction mixture at a concentration of about 1 mM to about 1000 mM, about 10 mM to about 990 mM, about 20 mM to about 980 mM, about 30 mM to about 970 mM, about 40 mM to about 960 mM, about 50 mM to about 950 mM, about 60 mM to about 940 mM, about 70 mM to about 930 mM, about 80 mM to about 920 mM, about 90 mM to about 910 mM, about 100 mM to about 900 mM, about 110 mM to about 890 mM, about 120 mM to about 880 mM, about 130 mM to about 870 mM, about 130 mM to about 860 mM, about 140 mM to about 850 mM, about 150 mM to about 840 mM, about 160 mM to about 830 mM, about 170 mM to about 820 mM, about 180 mM to about 810 mM, about 190 mM to about 800 mM, about 200 mM to about 790 mM, about 210 mM to about 780 mM, about 220 mM to about 770 mM, about 230 mM to about 760 mM, about 240 mM to about 750 mM, about 250 mM to about 740 mM, about 260 mM to about 730 mM, about 270 mM to about 720 mM, about 280 mM to about 710 mM, about 290 mM to about 700 mM, about 300 mM to about 690 mM, about 310 mM to about 680 mM, about 320 mM to about 670 mM, about 330 mM to about 660 mM, about 340 mM to about 650 mM, about 350 mM to about 640 mM, about 360 mM to about 630 mM, about 370 mM to about 620 mM, about 380 mM to about 610 mM, about 390 mM to about 600 mM, about 400 mM to about 590 mM, about 410 mM to about 580 mM, about 420 mM to about 570 mM, about 430 mM to about 560 mM, about 440 mM to about 550 mM, about 450 mM to about 540 mM, about 460 mM to about 530 mM, about 470 mM to about 520 mM, about 480 mM to about 510 mM, about 490 mM to about 510 mM, or about 500 mM.

In some embodiments, the composition comprises an isolated S. aureus PGN covalently bonded to an immunogenic polypeptide via a triazole moiety, wherein the PGN comprises an azide-modified D-amino acid and the immunogenic polypeptide is covalently bonded to a cycloalkyne moiety via a linker moiety.

In some embodiments, the reactive conjugation moiety is installed in the bacterial PGN for further modification via triazole formation with a suitably functionalized partner molecule (e.g., an immunogenic polypeptide with a complementary click functional group as described below). In such instances, the reactive conjugation moiety is a triazole precursor, such as an azide or an alkyne (e.g., a linear alkyne or a strained cycloalkyne such as a cyclooctyne).

In some embodiments, the reactive conjugation moiety comprises an azide having the formula —N₃. Azides can be bonded to the bacterial PGN directly or via linkers as described below. Azide-functionalized bacterial PGN can participate in click reactions such as the Huisgen 1,3-dipolar cycloaddition reaction. Azide groups may be incorporated into the PGN during growth of microbes in liquid culture as described below.

Nitrene groups, generated by expulsion of nitrogen gas upon exposure of azide-functionalized PGN to light or elevated temperatures, can also form covalent bonds via insertion into C—H bonds of an antigenic moiety (e.g., the immunogenic polypeptide) or other partner molecule.

In some embodiments, first or second reactive conjugation moiety comprises a cycloalkyne such as a cyclooctyne according to Formula I:

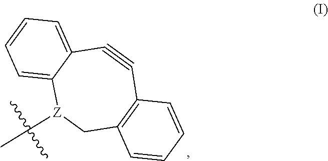

wherein the wavy line represents the point of connection to the immunogenic polypeptide or bacterial PGN, and Z is N or CH. Cyclooctynes according to Formula I can be bonded to the bacterial PGN directly or via a crosslinker reagent, e.g., linkers, as described below.

In some embodiments, the first or second reactive conjugation moiety comprises a linear alkyne such as a linear alkyne according to Formula II:

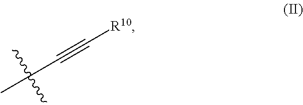

wherein the wavy line represents the point of connection to the immunogenic polypeptide or bacterial PGN, and R¹⁰ is selected from the group consisting of H and C₁₋₆ alkyl. Linear alkynes according to Formula II can be bonded to the bacterial PGN directly or via linkers as described below. Linear alkynes can react with azides via 1,3-dipolar cycloaddition reaction, which can be promoted by a copper-based catalyst such as a Cu(I) species (e.g., copper sulfate, copper acetate, copper triflate, or a copper halide) with or without a ligand such as a tris(triazolylmethyl)amine-based ligand. Ruthenium-based catalysts and other non-copper catalysts may also be employed. (see, for example, Singh et al. 2016. “Advances of azide-alkyne cycloaddition-click chemistry over the recent decade.” Tetrahedron 72: 5257-5283.doi.org/10.1016/j.tet.2016.07.044).

In some embodiments, the first or second reactive conjugation moiety comprises an aminooxy compound having the formula —ONH₂. Aminooxy compounds can be bonded to the bacterial PGN directly or via linkers as described below.

In some embodiments, the reactive conjugate moiety comprises a hydrazide having the formula —C(O)NHNH₂. Hydrazides can be bonded to the protein comprising the bacterial PGN directly or via linkers as described below.

In some embodiments, the reactive functional moiety comprises a ketone such as a ketone according to Formula IIIa:

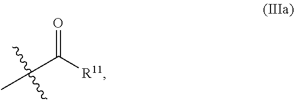

wherein the wavy line represents the point of connection to the immunogenic polypeptide or bacterial PGN, and R¹¹ is selected from the group consisting of C₁₋₆ alkyl, C₃₋₈ cycloalkyl, C₆₋₁₀ aryl, 5-to-12-membered heterocyclyl, and 5-to-12-membered heteroaryl. In some embodiments, R¹¹ is C₁₋₆ alkyl. Ketones according to Formula IIIa can be bonded to the bacterial PGN directly or via linkers as described below.

In some embodiments, the reactive functional moiety comprises an aldehyde such as an aldehyde according to Formula IIIb:

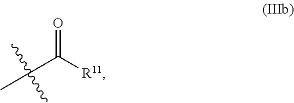

wherein the wavy line represents the point of connection to the immunogenic polypeptide or bacterial PGN, and R¹¹ is a hydrogen. Aldehydes according to Formula IIIb can be bonded to the bacterial PGN directly or via linkers as described below.

In some embodiments, the first or second reactive conjugation moiety comprises a phosphine such as a phosphine according to Formula IV:

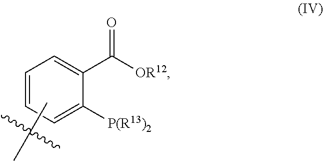

wherein the wavy line represents the point of connection to the immunogenic polypeptide or bacterial PGN; R¹² is independently selected from H and C₁₋₆ alkyl; and each R³ is independently selected from the group consisting of C₁₋₆ alkyl, C₃₋₈ cycloalkyl, 5-to-12-membered heterocyclyl, C₆₋₁₀ aryl, and 5-to-12-membered heteroaryl. In some embodiments, each R³ is phenyl. Phosphines according to Formula IV can be bonded to the bacterial PGN directly or via linkers as described below.

In some embodiments, first or second reactive conjugation moiety comprises a thiol having the formula —SH. Thiols can be bonded to the bacterial PGN directly or via linkers as described below. Thiols may also be present in sidechains of polypeptide cysteine residues.

In some embodiments, the first or second reactive conjugation moiety comprises a maleimide such as a maleimide according to Formula V:

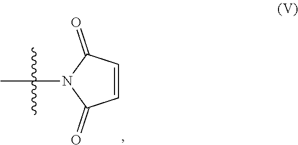 [SMILES: *N1C(=O)C=CC1=O]

wherein the wavy line represents the point of connection to the immunogenic polypeptide or bacterial PGN. Maleimides according to Formula V can be bonded to the immunogenic polypeptide or bacterial PGN directly or via linkers as described below.

A first reactive conjugation moiety of the bacterial PGN can be reacted with a complementary second reactive conjugation moiety on a suitably-functionalized immunogenic polypeptide to provide an immunogenic polypeptide conjugate. In some embodiments, the complementary second reactive conjugation moiety on the immunogenic polypeptide is selected from the group consisting of: a tetrazine a cycloalkyne (e.g., a cyclooctyne according to Formula I), a linear alkyne (e.g., a linear alkyne according to Formula U), an aminooxy compound, a hydrazide, a ketone (e.g., a ketone according to Formula III), an azide, a phosphine (e.g., a phosphine according to Formula IV), a thiol, and a maleimide (e.g., a maleimide according to Formula V). In general, the first reactive conjugation moiety of the bacterial PGN is different from the complementary second reactive conjugation moiety of the immunogenic polypeptide. In some embodiments, a linker moiety is present between the first reactive conjugation moiety and the PGN, and/or between the second reactive conjugation moiety and the immunogenic polypeptide. In some embodiments, for example, the linker moiety comprises an oligo(ethylene glycol) or a poly(ethylene glycol).

### Triazole Moiety

In some embodiments, the immunogenic polypeptide is bonded to the bacterial PGN via a triazole moiety, e.g., a triazole according to Formula VI:

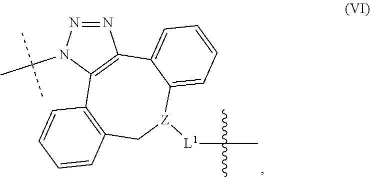

wherein
Z is CH or N,
L¹ is a linker moiety or a covalent bond,
the dashed line represents the point of connection to the bacterial PGN, and
the wavy line represents the point of connection to the immunogenic polypeptide.

In some embodiments, the triazole moiety is a reaction product formed via one or more click reactions between an azide-modified D-amino acid on the bacterial PGN and a cycloalkyne moiety on the immunogenic polypeptide.

### Azide

In some embodiments, the azide-modified D-amino acid is azido-D-alanine (azaDala).

### Cycloalkyne Moiety

In some embodiments, the cycloalkyne moiety is a cyclooctyne moiety such as, for example, a diarylcyclooctyne moiety (e.g., dibenzocyclooctyne (DBCO), azadibenzocyclooctyne (ADIBO), dibenzoazacyclooctyne (DIBAC), or (1R,8S,9S)-bicyclo[6.1.0]nonyne (BCN)). In some embodiments, the cyclooctyne moiety comprises DBCO. Cyclooctyne-functionalized immunogenic polypeptide can participate in click reactions such as Strain-Promoted Azide-Alkyne Cycloaddition (SPAAC), as known as copper-free click reaction, as discussed herein.

In the SPAAC reaction, a linear alkyne group is replaced with a cyclic analogue to react with azide in a more efficient manner, as a result of the high degree of ring strain of cycloalkyne. (Lu, et al., 2021). This method may be used for protein-nanoparticle conjugation and has high efficiency. SPAAC proceeds to a greater extent than N-hydroxysuccinimide (NHS) chemistry and can be further accelerated by modulating cycloalkyne structure, azide nature, and the solvent system used. For example, the use of dibenzoazacyclooctyne (DIBAC) or bicyclononyne (BCN) as the cycloalkyne-providing moiety in the acetonitrile-containing aqueous solvent increases reaction speed.

Unlike conventional click reactions, SPAAC is fast at room temperature and does not require a cytotoxic Cu(I) catalyst (that is toxic to most cellular organisms). Additionally. diarylcyclooctynes are thermostable with narrow and specific reactivity toward azides, resulting in quantitative yields of stable triazoles. Moreover, the strain-promoted or Cu(I)-free cycloaddition (SPAAC) strategy relies on the use of strained cyclooctynes. Accordingly, the use of diarylcyclooctynes decreases the activation energy for the cycloaddition click reaction, enabling it to be carried out without the need for catalysis at low temperatures with an efficiency greater than that of the Cu(I)-catalyzed ligation.

### Linker Moiety

In some embodiments, the cycloalkyne moiety is bonded to the immunogenic polypeptide via a linker moiety. Linker moieties according to the present disclosure join or connect a PGN sacculus and an antigen (e.g., immunogenic polypeptide) as provided herein. The linker moiety is typically flexible and may increase the range of orientations that may be adopted by the antigen with the respect to the PGN and/or the surrounding environment. In some instances, long soluble linkers (like PEG multimers) can help solubilize the immunogenic polypeptide and/or or other antigens attached to the PGN sacculus. Such linkers are usually readily amenable to conjugation to proteins. In some instances, longer linkers can be used to reach reactive functional groups deep within the PGN sacculus. Alternatively, shorter linkers can be used as they can produce less steric occlusion. In some instances, shorter linkers can allow for faster reaction times.

In some embodiments, the linking moiety L¹ of Formula VI has a structure -L^{1a}-L^{1b}-, wherein L^{1a} and L^{1b} are independently selected from a bond, a divalent polymer moiety, and linear or branched, saturated or unsaturated C₁₋₃₀ alkylene;
wherein one or more non-adjacent carbon atoms in the C₁₋₃₀ alkylene are optionally and independently replaced by O, S, NR^{a};
wherein one or more groupings of adjacent carbon atoms in the C₁₋₃₀ alkylene are optionally and independently replaced by —NR^{a}(CO)— or —(CO)NR^{a}—; and
wherein one or more groupings of adjacent carbon atoms in the C₁₋₃₀ alkylene are optionally and independently replaced by a 4- to 8-membered, divalent carbocycle or a 4- to 8-membered, divalent heterocycle having one to four heteroatoms selected from O, S, and N; and
wherein each Ra is independently selected from H and C₁₋₆ alkyl.

In some embodiments, the compositions provided herein comprise a peptide linker moiety. In some embodiments, the compositions provided herein comprise a non-peptide linker moiety. In some embodiments, the compositions provided herein comprise a peptide linker moiety and a non-peptide linker moiety. The proteins provided herein may also comprise a plurality of linker moieties, including at least one peptide linker moiety, at least one non-peptide linker moiety, or at least one peptide linker moiety and at least one non-peptide linker moiety. Different linker moieties in the plurality can be attached to different antigens (e.g., immunogenic polypeptides). A linker moiety may be flexible or rigid.

Non-peptide linker moieties of the provided compositions can comprise any of a number of known chemical linkers. Exemplary chemical linker moieties can include one or more units of beta-alanine, 4-aminobutyric acid (GABA), (2-aminoethoxy) acetic acid (AEA), 5-aminobexanoic acid (Ahx), polyethylene glycol (PEG) multimers, and trioxatricdeacan-succinamic acid (Ttds). In some embodiments, the non-peptide linker moiety comprises one or more units of PEG (i.e. PEG monomers or multimers), which is commonly used as a linker moiety for conjugation of polypeptide domains due to its water solubility, lack of toxicity, low immunogenicity, and well-defined chain lengths. See, for example, e.g., Ramirez-Paz, J., et al., PLoS One 13(7):e0197643 (2018). The number of PEG linkage units may be selected based on the desired length of the linker moiety. In some embodiments, the linker moiety comprises one or more ethylene glycol moieties. In some embodiments, the linker moiety comprises PEG3, PEG4, or PEG8.

In some instances, a peptide linker moiety may be used. Depending on length, the peptide linker moiety may have various conformations in secondary structure, such as helical, β-strand, coil/bend, and turns. Flexible peptide linker moieties provide a certain degree of movement or interaction between the PGN sacculus and the antigen (e.g., immunogenic polypeptide) and are generally rich in small or polar amino acids such as Gly and Ser (e.g., at least 90%, at least 95%, at least 98%, at least 99%, or all of the amino acid residues of the linker are either Gly or Ser). A rigid peptide linker moiety can be used to keep a fixed distance between the domains and to help maintain their independent functions. Linker moiety attachment can be through an amide linkage (e.g., a peptide bond) or other functionalities as discussed further below.

In some embodiments, the linker moiety is installed via reaction of an amino acid residue in the immunogenic polypeptide and a crosslinker reagent comprising the cycloalkyne moiety and a peptide-reactive handle.

Alternatively, the linker moiety can be first installed via reaction of an amino acid residue in the PGN (e.g., an azide-containing amino acid) and a crosslinker reagent comprising the cycloalkyne moiety and a peptide-reactive handle (e.g., a maleimide moiety). In a second step, the PGN having the appended maleimide group can then be reacted with the immunogenic polypeptide having a reactive amino acid residue (e.g., a cysteine residue).

Examples of suitable crosslinker reagents include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (sulfhydryl reactive); aryl azides (primary amine reactive); fluorinated aryl azides (reactive via carbon-hydrogen (C—H) insertion); pentafluorophenyl (PFP) esters (amine reactive); imidoesters (amine reactive); isocyanates (hydroxyl reactive); vinyl sulfones (sulfhydryl, amine, and hydroxyl reactive); pyridyl disulfides (sulfhydryl reactive); and benzophenone derivatives (reactive via C—H bond insertion). Further reagents include, but are not limited to, those groups and methods described in Hermanson, Bioconjugate Techniques 2nd Edition, Academic Press, 2008.

### Crosslinker Reagent

In certain embodiments, the crosslinker reagent comprises one or more ethylene glycol moieties. In some embodiments, the crosslinker reagent is a polyethylene glycol (PEG), also referred to as polyethylene oxide (PEO) or polyoxyethylene (POE). In various embodiments, the polyethylene glycol is a PEG3, PEG4, or PEG8. In some embodiments, the polyethylene glycol is PEG4.

In some embodiments, the crosslinker reagent has a structure selected from:

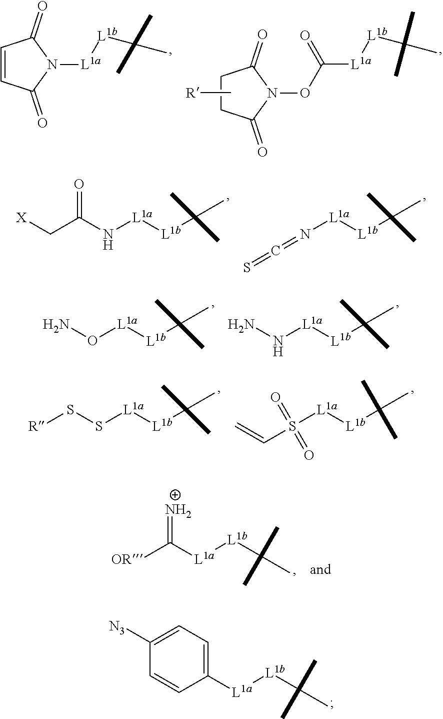

wherein X is halogen (e.g., iodo or chloro); R′ is H or sulfo; R″ is optionally substituted aryl (e.g., 3-carboxy-4-nitrophenyl) or optionally substituted heteroaryl (e.g., pyridin-2-yl); R′″ is optionally substituted alkyl (e.g., methoxy); L^{1a} and L^{1b} are as described above; and the bold line represents the point of connection to a click moiety (e.g., to L^{1a}-L^{1b} corresponds to L¹ of Formula VI above, wherein the bold line represents the point of connection to Z of Formula VI).

In some embodiments, the peptide-reactive handle is a maleimide, which can be used for reaction with a cysteine residue in the immunogenic polypeptide. In some embodiments, the peptide-reactive handle is a N-hydroxysuccinimidyl ester (NHS ester), which can be used for reaction with a lysine residue in the immunogenic polypeptide.

Maleimide-mediated click reactions are widely used in bioconjugation. Due to exceptionally fast reaction rates and significantly high selectivity towards cysteine residues in proteins, a large variety of maleimide heterobifunctional reagents are used in biotechnology applications. Maleimides linked to polyethylene glycol chains are often used as flexible linking molecules to attach proteins to surfaces. The double bond readily reacts with the thiol group found on cysteine to form a stable carbon-sulfur bond. Attaching the other end of the polyethylene chain to a bead or solid support allows for easy separation of protein from other molecules in solution, provided these molecules do not also possess thiol groups. For instance, as described herein, maleimide peptide-reactive handles are used to conjugate an immunogenic polypeptide to a modified S. aureus PGN comprising an azide-modified D-amino acid (i.e., azido-D-alanine (azaDala)). In some embodiments, the crosslinker reagent comprises a maleimide peptide-reactive handle and a cycloalkyne moiety. For example, the crosslinker reagent may be:

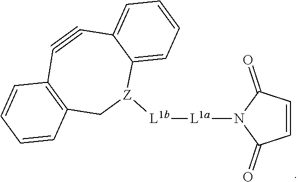

N-hydroxysuccinimide (NHS) esters can react with amines at pH 7-9, even without carbodiimide pre-activation. NHS esters can also react with serine, tyrosine, and threonine hydroxyl residues in proteins, forming undesired conjugation sites. NHS esters may be introduced to the conjugation media as aliquots in organic solvent, such as DMSO or DMF. To preserve the structure and functionality of the proteins, the amount of organic solvent in the final conjugation media generally does not exceed a pre-determined percentage (e.g., 10%). In some instances, NHS ester comprises a charged sulfonate group for increasing water solubility. In some instances, sodium phosphate buffer (0.1 M) and NaCl (0.15 M) at pH 7.2-7.5 as a solvent for this conjugation method. In some embodiments, the crosslinker reagent comprises an NHS ester peptide-reactive handle and a cycloalkyne moiety. For example, the crosslinker reagent may be:

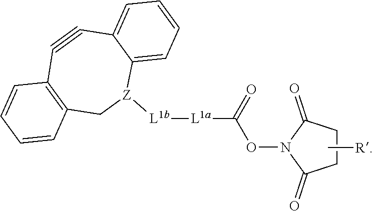

Crosslinker reagents for functionalization of PGNs and/or immunogenic polypeptides are typically employed using the general reaction conditions described above with respect to click conjugation. In some embodiments, an excess of the crosslinker reagent (e.g., 2 molar equivalents, 5 molar equivalents, 25 molar equivalents, 50 molar equivalents, or more) with respect to the PGN and/or the immunogenic polypeptide is employed. Excess reagents may be removed if appropriate (e.g., via gel filtration or a like technique) prior to conducting the click conjugation chemistry in subsequent steps.

### Additional Click Conjugates

As described above, other pairs of reactive functional groups may be used for conjugation in place of the azide and the cyclooctyne, providing conjugates with various connecting linkages. Some embodiments of the present disclosure comprise conjugates wherein the immunogenic polypeptide is bonded to the bacterial PGN via a hydrazide moiety, e.g., a hydrazide according to Formula VIIa or Formula VIIb:

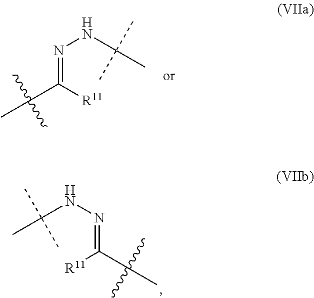

wherein
the wavy line represents the point of connection to the bacterial PGN or the immunogenic polypeptide,
the dashed line represents the point of connection to the immunogenic polypeptide or the bacterial PGN, and
R¹¹ is selected from the group consisting of H, C₁₋₆ alkyl, C₃₋₈ cycloalkyl, 5-to-12-membered heterocyclyl, C₆₋₁₀ aryl, and 5-to-12-membered heteroaryl.

Some embodiments of the present disclosure comprise conjugates wherein the immunogenic polypeptide is bonded to the bacterial PGN via an oxime moiety, e.g., an oxime according to Formula Villa or Formula VIIIb:

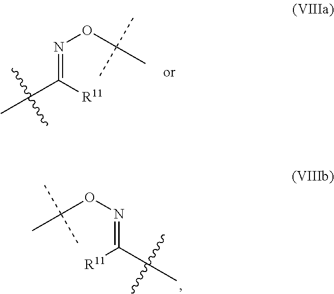

wherein
the wavy line represents the point of connection to the bacterial PGN or the immunogenic polypeptide,
the dashed line represents the point of connection to the immunogenic polypeptide or the bacterial PGN, and
R¹¹ is selected from the group consisting of H, C₁₋₆ alkyl, C₃₋₈ cycloalkyl, 5-to-12-membered heterocyclyl, C₆₋₁₀ aryl, and 5-to-12-membered heteroaryl.

Some embodiments of the present disclosure comprise conjugates wherein the immunogenic polypeptide is bonded to the bacterial PGN via an amide moiety, e.g., a phosphoryl-substituted amide according to Formula IXa or Formula IXb:

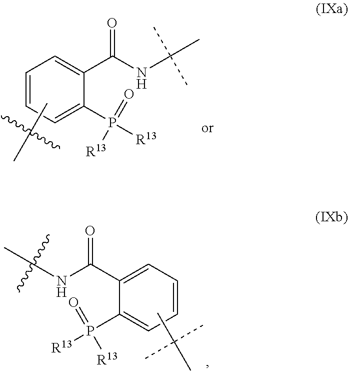

wherein
the wavy line represents the point of connection to the bacterial PGN or the immunogenic polypeptide,
the dashed line represents the point of connection to the immunogenic polypeptide or the bacterial PGN, and
each R¹³ is independently selected from the group consisting of C₁₋₆ alkyl, C₃₋₈ cycloalkyl, 5-to-12-membered heterocyclyl, C₆₋₁₀ aryl, and 5-to-12-membered heteroaryl.

Some embodiments of the present disclosure comprise conjugates wherein the immunogenic polypeptide is bonded to the bacterial PGN via a thioether moiety, e.g., a thioether according to Formula Xa or Formula Xb:

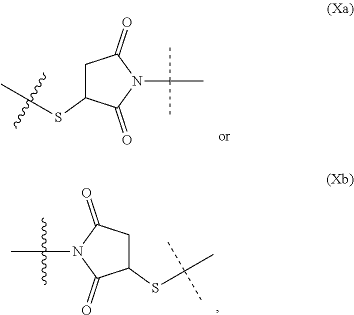

wherein
the wavy line represents the point of connection to the bacterial PGN or the immunogenic polypeptide, and
the dashed line represents the point of connection to the immunogenic polypeptide or the bacterial PGN.

Reactive functional groups in conjugates, crosslinkers, functionalized PGNs, and functionalize immunogenic polypeptides according to the present disclosure may be optionally substituted with further functional groups. In general, the term “substituted,” whether preceded by the term “optionally” or not, means that one or more hydrogen atoms in a designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents are generally those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. In general, “substituted,” as used herein, does not encompass replacement and/or alteration of a key functional group by which a molecule is identified, e.g., such that the “substituted” functional group becomes, through substitution, a different functional group. For example, a “substituted phenyl” group must still comprise the phenyl moiety and cannot be modified by substitution, in this definition, to become, e.g., a cyclohexyl group.

### IV. ADJUVANTS AND STABILIZING AGENTS

As discussed above, the compositions provided herein are useful as subunit vaccines, for example. In the context of a subunit vaccine, adjuvants are generally defined as substances that increase immunogenicity of a vaccine formulation when added or mixed to it. (Juliana de Souza et al., 2016. “Adjuvants: Classification, Modus Operandi, and Licensing”, Journal of Immunology Research, Article ID 1459394.doi.org/10.1155/2016/1459394). Given that PGN sacculi can be used as microparticle carriers, the compositions provided herein can readily be combined with a variety of other adjuvants, for instance, vaccine platforms including alum, emulsions, liposomes, polymeric particles (PLG), and particulate systems, further increasing their immunogenicity. In some instances, an adjuvant can be combined with the compositions described above, for example, as a component in a formulation. In some instances, the adjuvant can be covalently attached to the surface of the PGN sacculus. In some instances, a plurality of adjuvants may be used, one or more covalently attached to the surface of the PGN sacculus and one or more combined with the compositions described above. In some instances, multiple adjuvants can be tethered together, for example linking multiple TLR agonists together better stimulates the immune response than untethered versions. (Tom et al., 2015. “Modulation of Innate Immune Responses via Covalently Linked TLR Agonists.” ACS Cent. Sci., 1(8), 439-448.doi.org/10.1021/acscentsci.5b00274).

There are a variety of adjuvants conventionally used for increasing immunogenicity of a vaccine formulation. Conventional adjuvants may include chemical adjuvants, genetic adjuvants, and protein adjuvants. Some examples of chemical adjuvants include mineral salts (e.g., aluminum salts or alum, calcium phosphate), aluminum phosphate, benzyalkonium chloride, ubenimex, QS21, aluminum hydroxide (such as alum, an aluminum hydroxide wet gel suspension, for example, Alhydrogel® (Croda International, UK)), saponins (for example, Quil-A® (Croda International, UK)), squalenes (for example, AddaVax™). Some examples of the so-called “genetic” adjuvants are IL-2 gene or its fragments, granulocyte macrophage colony-stimulating factor (GM-CSF) gene or fragments thereof, IL-18 gene or fragments thereof, chemokine (C—C motif) ligand 21 (CCL21) gene or fragments thereof, IL-6 gene or fragments thereof, CpG, LPS, TLR agonists (e.g., Monophosphoryl Lipid A (MPLA)), and other immune stimulatory genes. Some examples of protein adjuvants are IL-2 or fragments thereof, granulocyte macrophage colony-stimulating factor (GM-CSF) or fragments thereof, IL-18 or its fragments, chemokine (C—C motif) ligand 21 (CCL21) or fragments thereof, IL-6 or fragments thereof, CpG, LPS, TLR agonists, T-cell epitopes, and other immune stimulatory cytokines or their fragments. Some examples of lipid adjuvants are cationic liposomes, N3 (cationic lipid), MPLA, Quil-A®, and AddaVax™. Other exemplary adjuvants include, but are not limited to, cholera toxin, enterotoxin, Fms-like tyrosine kinase-3 ligand (Flt-3L), bupivacaine, marcaine, and levamisole. In some embodiments, the composition comprises Quil-A®. Other exemplary adjuvants include, but are not limited to, emulsions or lipid particles (e.g., Incomplete Freund's adjuvant (IFA), MF59, cochleates), microparticles (e.g., virus-like particles VLPs), virosomes, poly(lactic acid) (PLA), poly(lactic-coglycolic acid) (PLGA)), immune potentiators (e.g., dsRNA: Poly(I:C), Poly-IC:LC, monophosphoryl lipid A (MPL), lipopolysaccharide (LPS), flagellin, imidazoquinolines such as imiquimod (R837), resiquimod (848), Muramyl dipeptide (MDP), CpG oligodeoxynucleotides (ODN), Saponins, mucosal adjuvants (e.g., cholera toxin (CT), heat-labile enterotoxin (LTK3 and LTR72), chitosan), cytokines, microbial components/products, and liposomes.

In some instances, adjuvants can increase the biological half-life of vaccines, increase antigen uptake by antigen presenting cells (APCs), activate/mature APCs (e.g., dendritic cells), induce the production of immunoregulatory cytokines, activate inflammasomes, and induce local inflammation and cellular recruitment. In some embodiments, the adjuvant can be a membrane protein that play a role in the innate immune system. For example, the adjuvant can be a Toll-like receptor agonist. In some embodiments, the adjuvant can be an epitope presented on the surface of an antigen-presenting cell (APC). For example, the adjuvant can be a T-cell epitope.

More than one adjuvant may be included in compositions according to the embodiments of the present disclosure. For example, in some embodiments, the composition can comprise alum and CpG. In some embodiments, the composition can comprise a Toll-like receptor agonist (TLR) and a T-cell epitope. In some embodiments, the composition can comprise one or more Toll-like receptor agonist (TLR), one or more T-cell epitope, and/or one and more alum, and/or CpG, and/or combinations thereof.

In some instances, the adjuvant combined with or covalently attached to the compositions provided herein can be a T-cell epitope. T cells recognize antigens as peptides associated with self molecules encoded by major histocompatibility (MHC) molecules and presented on the surface of an antigen-presenting cell (APC). Thus, the binding of immunogenic peptides to MHC molecules is a prerequisite for T cell activation. T-cell epitopes are typically peptide fragments. In humans, professional antigen-presenting cells (APC) are specialized to present MHC class 11 peptides, whereas most nucleated somatic cells present MHC class I peptides. T cell epitopes presented by MHC class I molecules are typically peptides between 8 and 11 amino acids in length, whereas MHC class II molecules present longer peptides, 12-25 amino acids in length, and non-classical MHC molecules also present non-peptidic epitopes such as glycolipids. MHC class II proteins bind oligopeptide fragments derived through the proteolysis of pathogen antigens, and present them at the cell surface for recognition by CD4′ T cells. If sufficient quantities of the epitope are presented, the T cell may trigger an adaptive immune response specific for the pathogen. T cell responses are generally specific for a few, and often only one, of the peptides derived by processing of a protein antigen. These T cell epitopes are referred to as immunodominant (i.e. are immunodominant target antigens). As used herein, the term “T-cell epitope” refers to an immunodominant T cell epitope that can induce a specific immune response.

In some instances, the adjuvant combined with or covalently attached to the compositions provided herein can be a Toll-like receptor agonist. Toll-like receptors (TLRs) are a class of proteins that play a key role in the innate immune system. TLRs are single-pass membrane-spanning receptors usually expressed on expressed on innate immune cells (sentinel cells) such as dendritic cells (DCs) and macrophages as well as non-immune cells such as fibroblast cells and epithelial cells. TLRs play crucial roles in the innate immune system by recognizing pathogen-associated molecular patterns derived from various microbes. TLRs are a type of pattern-recognition receptors (PRPs) that recognize microbe-specific molecular signatures known as pathogen-associated molecular patterns (PAMPs) and self-derived molecules that lead to the induction of innate immune responses by producing inflammatory cytokines, type I interferon (IFN), and other mediators. These processes trigger immediate host defensive responses such as inflammation and orchestrate antigen-specific adaptive immune responses. These responses are essential for the clearance of infecting microbes as well as crucial for the consequent instruction of antigen-specific adaptive immune responses. Toll-like receptors (TLRs) are TLRs are largely classified into two subfamilies based on their localization, cell surface TLRs and intracellular TLRs. Cell surface TLRs include TLR1, TLR2, TLR4, TLR5, TLR6, and TLR10, whereas intracellular TLRs are localized in the endosome and include TLR3, TLR7, TLR8, TLR9, TLR11, TLR12, and TLR13. Human lacks TLR1, TLR12, and TLR13.

The use of potent TLR ligands to activate TLR signaling cascades allows specific TLRs and their effect on immune activation to be studied. Exemplary TLR agonists are provide in Table 2. (See novusbio.com/immunology/toll-like-receptor-agonists).

TABLE 2
Exemplary Toll-like receptor and agonists.

| Toll-like | | |
|---|---|---|
| Receptor | Ligand | Description |
| TLR1 | PAM3CSK4 | A synthetic lipopeptide that mimics the acylated amino terminus of |
| | | bacterial lipoproteins. It is recognized by a heterodimer which is |
| | | formed between TLR1 and TLR2. |
| TLR2 | PAM3CSK4 | A synthetic lipopeptide that mimics the acylated amino terminus of |
| | | bacterial lipoproteins. It is recognized by a heterodimer which is |
| | | formed between TLR1 and TLR2. |
| | MALP-2 | A lipopeptide originally isolated from Mycoplasma fermentans. |
| | | MALP-2 is recognized by a heterodimer formed between TLR2 |
| | | and TLR6. |
| | Zymosan | A cell wall preparation of Saccharomyces cerevisiae and is |
| | | composed primarily of glucans, mannans, mannoproteins and |
| | | chitin. It activates TLR2 in synergistic collaboration with Dectin-1, |
| | | a C-type lectin that is expressed on monocytes, macrophages, and |
| | | dendritic cells as a phagocytic receptor for glucan containing |
| | | particles. |
| TLR3 | Poly (I:C) | A synthetic analogue of double-stranded RNA, produced during |
| | | the replication of many viruses; it binds directly to TLR3. |
| TLR4 | LPS | A standard lipopolysaccharide preparation. LPS is a major |
| | | component of the cell wall of Gram-negative bacteria. Recognition |
| | | of LPS requires TLR4 in addition to CD14, and is enhanced by |
| | | MD-2, a secreted glycoprotein which forms a receptor complex |
| | | with TLR4. |
| TLR5 | Flagellin | A key component of bacterial flagellae, which are appendages used |
| | | primarily for locomotion. It is highly conserved amongst both |
| | | Gram-positive and Gram-negative bacteria, and is recognized by |
| | | TLR5. |
| TLR6 | MALP-2 | A lipopeptide originally isolated from Mycoplasma fermentans. |
| | | MALP-2 is recognized by a heterodimer formed between TLR2 |
| | | and TLR6. |
| TLR7 | Imiquimod | An imidazoquinoline amine analog to guanosine. |
| | Imidazoquinoline | A synthetic imidazoquinoline. It has been demonstrated to activate |
| | Resiquimod | TLR7 and TLR8. |
| TLR8 | Imidazoquinoline | A synthetic imidazoquinoline. It has been demonstrated to activate |
| | Resiquimod | TLR7 and TLR8. |
| TLR9 | CpG ODN | Synthetic oligonucleotide containing unmethylated deoxycytosine- |
| | | deoxyguanosine (CpG) motifs. |

Given that PGN sacculi can be used as microparticle carriers, the compositions provided herein can readily be combined with a variety of stabilizing agents. The addition of a stabilizing agent to the compositions described herein can further increase the stability of the active agent, i.e., the active agent can retain a higher bioactivity, relative to the bioactivity in the absence of the stabilizing agent. In some instances, an adjuvant can be combined with the compositions described above, for example, as a component in a formulation. In some instances, the adjuvant can be covalently attached to the surface of the PGN sacculus. In some instances, a plurality of adjuvants may be used, one or more covalently attached to the surface of the PGN sacculus and one or more combined with the compositions described above.

In some embodiments, the stabilizing agent can be a saccharide, a sugar alcohol, an ion, a surfactant, and any combinations thereof. Exemplary stabilizing agents include cationic stabilizers (listed most to least stabilizing): (CH3)₄N*>Mg2+, K+>Na*, NH 4+>Li+; anionic stabilizers (most to least stabilizing): CH3COO—, SO4-, P0 4 2->Cl—, SCN—; and heavy water (D20); amino acids such as sodium glutamate, arginine, lysine, and cysteine; monosaccharides, such as glucose, galactose, fructose, and mannose; disaccharides, such as sucrose, maltose, and lactose; sugar alcohols such as sorbitol and mannitol; polysaccharides, such as oligosaccharide, starch, cellulose, and derivatives thereof; human serum albumin and bovine serum albumin; gelatin, and gelatin derivatives, such as hydrolyzed gelatin; and ascorbic acid as an antioxidant. In one embodiment, the saccharide, e.g., sucrose, is added into the compositions described herein. In some embodiments, the stabilizing agent is a silk fibroin matrix as described in U.S. Application No: 20170258889. In some embodiments, the stabilizing agent is an injectable polymer-nanoparticle hydrogel (Brito et al., 2013, Seminars in Immunology 25(2), 130-145).

### V. FORMULATIONS

Provided herein are formulations that comprise compositions as described the present disclosure and a pharmaceutically acceptable excipient. In some embodiments, the formulation can further comprise an adjuvant and/or a stabilizing agent as described above in Section IV.

A pharmaceutically acceptable carrier or excipient is a material that is not biologically or otherwise undesirable, meaning the material that can be administered to a subject without causing undesirable biological effects or interacting in a deleterious manner with the other components of the pharmaceutical composition in which it is contained. The carrier or excipient is typically selected to minimize degradation of other ingredients of the composition in which the carrier or the excipient is included, and to minimize adverse side effects (such as allergic side effects) in the subject. Examples of aqueous pharmaceutically acceptable carriers include, but are not limited to, sterile water, saline, buffered solutions like Ringer's solution, glycerol solutions, ethanol, dextrose solutions, allantoic fluid, or combinations of the foregoing. The pH of the aqueous carriers is generally about 5 to about 8 or from about 7 to 7.5. A carrier may include a pH-controlling buffer. The preparation of such aqueous carriers insures sterility, pH, isotonicity, and stability is not affected according to established protocols. Examples of non-aqueous carriers are propylene glycol, polyethylene glycol, vegetable oils such as olive oil, and injectable organic esters such as ethyl oleate. Other exemplary carriers include sustained release preparations, such as semipermeable matrices of solid hydrophobic polymers. Other exemplary carriers are matrices in the form of shaped articles, such as, but not limited to, films, liposomes, or microparticles. Certain carriers may be more preferable depending upon, for instance, the composition, the route of administration, and the concentration of composition being administered.

Formulations according to the embodiments of the present disclosure are generally formulated to be nontoxic or minimally toxic to a/the subject at the dosages and concentrations used for administration. In some embodiments, a formulation of a compositions includes an appropriate amount of a pharmaceutically acceptable salt to render the formulation isotonic. In some embodiments, a formulation of a compositions includes components for modifying, maintaining, or preserving, for example, the pH, osmolality, viscosity, clarity, color, isotonicity, odor, sterility, stability, rate of dissolution or release, adsorption or penetration of the composition. A formulation of a composition may include one or more of the following components: amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); and/or delivery vehicles.

In some embodiments, the composition is prepared in a dry form (i.e. dehydrated form), such as a lyophilized form. Such a formulation can be referred to as “lyophilized” or a “lyophilizate.” Lyophilization is a process of or freeze-drying, during which a solvent is removed from a liquid formulation. Lyophilization process may include one or more of simultaneous or sequential steps of freezing and drying. Compositions according to the embodiments of the present disclosure can be lyophilized in an aqueous solution comprising a nonvolatile or volatile buffer. Non-limiting examples of suitable nonvolatile buffers are PBS, Tris-HCl, HEPES, or L-Histidine buffer. Non-limiting examples of suitable volatile buffers are ammonium bicarbonate, Ammonia/acetic acid, or N-ethylmorpholine/acetate buffer. A lyophilized composition according to the embodiments of the present disclosure can include appropriate carriers or excipients. Such appropriate excipients may include, but are not limited to, a cryo-preservative, a bulking agent, a surfactant, or their combinations. Exemplary excipients include one or more of a polyol, a disaccharide, or a polysaccharide, such as, for example, mannitol, sorbitol, sucrose, trehalose, and/or dextran 40. In some instances, the cryo-preservative may be sucrose and/or trehalose. In some instances, the bulking agent may be glycine or mannitol. In one example, the surfactant may be a polysorbate such as, for example, polysorbate-20 and/or polysorbate-80. A lyophilized composition according to the embodiments of the present disclosure can be, for example, in a cake or powder form. Lyophilized compositions may be rehydrated/solubilized/reconstituted in a carrier or excipient (e.g., water or buffer solution) prior to use. Some embodiments of the compositions are reconstituted in a water or buffer solution comprising sucrose.

Compositions according to embodiments of the present disclosure can be sterile prior to administration to a subject. Sterilization can be accomplished, for example, by filtration through sterile filtration membranes or other methods known in the art. When the composition is lyophilized, sterilization can be conducted either prior to or following lyophilization and reconstitution. The composition can be stored in sterile containers, such as vials or bags, as a solution, suspension, gel, emulsion, solid, or as a dehydrated or lyophilized powder.

### VI. KIT

Kits including compositions described in the present disclosure are also included among the embodiments of the present disclosure. For example, a kit may include a composition and a container for its storage, such as a bag or a vial. Such a container may have a sterile access port, for example, a bag or vial having a stopper pierceable by a hypodermic injection needle. In another example, a kit may include a composition in lyophilized or concentrated form. In another example, a kit may include a composition in lyophilized or concentrated form and diluent. In such a kit, a diluent may also be a pharmaceutically acceptable carrier or excipient, as described elsewhere in the present disclosure. Examples of diluents that may be included in such a kit are saline, buffered saline, water, or sucrose. In another example, a kit may include a composition and a device for administering the composition. A device for administering the composition may be a syringe for injection or oral administration (for example, the kit may be a syringe pre-filled with a liquid composition), a microneedle device, such as a microneedle patch, an inhaler, or a nebulizer. For example, a kit may contain multiple vials, syringes or microneedle patches containing a composition. Such administration devices may be single use (i.e., disposable) or reusable.

In some embodiments, a kit contains a defined amount of a composition capable of eliciting a protective immune response against a coronavirus in a subject, when administered as a single dose. In some embodiments, a kit contains multiple doses of a defined amount of a composition capable of eliciting a protective immune response against a coronavirus in a subject.

In some embodiments, a kit contains a defined amount of a composition capable of eliciting a protective immune response against a cancer in a subject, when administered as a single dose (also referred to herein as “an effective amount”). In some embodiments, a kit contains multiple doses of a defined amount of a composition capable of eliciting a protective immune response against a cancer in a subject.

### VII. METHODS

Provided are methods of inducing or eliciting an immune response in a subject by administering to the subject a composition or formulation described herein. In embodiments of such methods, the composition is administered to the subject in an amount capable of inducing or eliciting a protective immune response against the immunogenic polypeptide of the composition in the subject; particularly, against the microorganism or cell that expresses the immunogenic polypeptide. For instance, a protective immune response against a pathogen in the subject may include production in the subject of neutralizing antibodies that bind specifically to the immunogenic polypeptide of the provided compositions. In some embodiments, the subject has a SARS-CoV-2 infection. In some embodiments, the subject is suspected of having a SARS-CoV-2 infection (e.g., the subject has contact with someone who has been confirmed or diagnosed of having a SARS-CoV-2 infection). In some embodiments, the subject is at risk of exposure to SARS-CoV-2 infection, e.g., the subject the subject has a higher probability of exposing to SARS-CoV-2 infection due to the work or living environment. An amount of the composition capable of inducing or eliciting a protective immune response in a subject can be described as an “pharmaceutically effective amount” or “immunologically effective amount,” both considered to be an “effective amount” within the context of this disclosure, and may be administered as one dose or as two or more doses. Generally, an immunogenically effective amount refers to an amount that induces an immune response, e.g., antibodies (humoral) against the immunogenic polypeptide when administered to a subject. The compositions may also induce cellular (non-humoral) immune responses. Effective amounts and schedules for administration may be determined empirically.

In some embodiments, the subject is administered more than one dose of the composition over a period of time. For example, the subject may be administered a first effective dose (or effective amount) of the composition on day 1, followed by a second effective dose of the composition on day 28. The subject may be administered a third effective dose of the composition on day 112. In some embodiments, the first effective dose of the composition is administered on day 1 and the one or more subsequent effective doses of the composition are administered on about day 28, about day 112, about day 150, about day 180, about day 360, or longer. In some instances, the subsequent doses are booster doses having the same dosage as the first effective dose. In some instances, the subsequent doses are booster doses having a lower or a higher dosage in reference to the first effective dose.

Dosage ranges for administration of the compositions described in the present disclosure are those large enough to produce the desired effect—i.e. eliciting a protective immune response against the immunogenic polypeptide of the composition in the subject; particularly, against the microorganism or cell that expresses the immunogenic polypeptide. The dosage should not be so large as to cause substantial adverse side effects, such as unwanted cross-reactions, anaphylactic reactions, and the like. Generally, the dosage may vary with the age, condition, sex, medical status, route of administration, or whether other drugs are included in the regimen. The dosage can be adjusted by a medical professional in the event of any contraindications. Dosages can vary, and the agent can be administered in one or more dose administrations daily, for one or several days, including a prime and boost paradigm.

The compositions and formulations described herein can be administered via any of several routes of administration, including, but not limited to, orally, parenterally, intravenously, intramuscularly, subcutaneously, transdermally, by nebulization/inhalation, or by installation via bronchoscopy. Administration can be by oral inhalation, nasal inhalation, or intranasal mucosal administration. Administration by inhalant can be through the nose or mouth via delivery by spraying or droplet mechanism, for example, in the form of an aerosol. In some embodiments, administration is intramuscular or subcutaneous. A form of administration may be chosen to optimize a protective immune response against a coronavirus or a cancer in a subject.

As used throughout, by “subject” is meant an individual. For example, the subject is a mammal, such as a primate, and, more specifically, a human. Non-human primates can be subjects according to the present disclosure as well. The term “subject” includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations for non-humans are contemplated herein. The term does not denote a particular age or sex. Thus, adult and newborn subjects, whether male or female, are intended to be covered. As used herein, patient or subject may be used interchangeably and can refer to a subject afflicted with a disease or disorder.

In some embodiments, the immunogenic polypeptide of the provided composition is a viral protein such as a coronavirus protein. In the context of the methods described in the present disclosure, a subject may be healthy and without higher risk for a coronavirus infection than the general public. In some instances, the subject can have an elevated risk of developing a coronavirus infection such that they are predisposed to contracting an infection, or may be predisposed to developing a serious form of coronavirus disease, such as COVID-19 (for example, persons over 65, persons with asthma or other chronic respiratory disease, young children, pregnant women, persons with a hereditary predisposition, persons with a compromised immune system may be predisposed to developing a serious form of COVID-19). A subject may also be a subject with a current coronavirus infection and may have one or more than one symptom of the infection. A subject currently with a coronavirus infection may have been diagnosed with coronavirus infection based on the symptoms or the results of diagnostic tests.

In some embodiments, the immunogenic polypeptide of the provided composition is a cancer neoantigen protein. In some instances, the subject can have an elevated risk of developing a cancer such that they are predisposed to developing a cancer, or may be hereditary predisposed, such as mutation of the gene HER-2. A subject may also be a subject with a current cancer. A subject may also be a subject who have had a cancer and is currently in remission, or has no evidence of disease based on the results of diagnostic tests. A subject may also be a subject with symptoms of cancer or diagnosis of cancer but is potentially susceptible of developing a cancer based on familiar history and/or the results of genetic screenings.

The methods according to the embodiments of the present disclosure are useful for both prophylactic and therapeutic purposes. Methods of treating or preventing an infection in a subject, which include administering to a subject with an infection or susceptible to an infection an effective dose of compositions or formulations described herein are also included among the embodiments of the present disclosure. In some embodiments, a composition or formulation as described herein can be used alone or in combination with one or more therapeutic agents such as, for example, antiviral compounds for the treatment of a viral infection or disease. For prophylactic use, an effective amount of a composition or formulation described herein can be administered to a subject prior to onset of an infection (for example, before obvious signs of infection) or during early onset (for example, upon initial signs and symptoms of infection). Prophylactic administration can occur at several days to years prior to the manifestation of symptoms of coronavirus infection. Prophylactic administration can be used, for example, in the preventative treatment of subjects identified as having a predisposition to an infection. Therapeutic treatment involves administering to a subject a therapeutically effective amount of a composition described in the present disclosure after diagnosis or development of infection.

Methods of treating cancer in a subject, which include administering to a subject with cancer or susceptible to developing a cancer an effective dose compositions described herein, are also included among the embodiments of the present disclosure. The provided compositions and formulations can be used alone or in combination with one or more therapeutic agents.

In some embodiments, the methods provided herein can further comprise administering to the subject one or more additional therapies. For example, in methods of treating an infection in a subject, suitable additional types of therapies include anti-viral agents and antibiotics. For example, in methods of treating cancer in a subject, suitable additional types of therapies include, but are not limited to, chemotherapy, immunotherapy, radiotherapy, hormone therapy, differentiating agents, and small-molecule drugs. One of skill in the art will readily be able to select an appropriate additional therapy.

Chemotherapeutic agents that can be used in the present disclosure include but are not limited to: alkylating agents (e.g., nitrogen mustards (e.g., mechlorethamine, chlorambucil, cyclophosphamide, ifosfamide, melphalan), nitrosoureas (e.g., streptozocin, carmustine (BCNU), lomustine), alkyl sulfonates (e.g., busulfan), triazines (e.g., dacarbazine (DTIC), temozlomide), ethylenimines (e.g., thiotepa, altretamine (hexamethylmelamine))), platinum drugs (e.g., cisplatin, carboplatin, oxalaplatin), antimetabolites (e.g., 5-fluorouracil (5-FU), 6-mercaptopurine (6-MP), capecitabine, cytarabine, floxuridine, fludarabine, gemcitabine, hydroxyurea, methotrexate, pemetrexed), anthracycline anti-tumor antibiotics (e.g., daunorubicin, doxorubicin, epirubicin, idarubicin), non-anthracycline anti-tumor antibiotics (e.g., actinomycin-D, bleomycin, mitomycin-C, mitoxantrone), mitotic inhibitors (e.g., taxanes (e.g., paclitaxel, docetaxel), epothilones (e.g., ixabepilone), vinca alkaloids (e.g., vinblastine, vincristine, vinorelbine), estramustine), corticosteroids (e.g., prednisone, methylprednisolone, dexamethasone), L-asparaginase, bortezomib, and topoisomerase inhibitors. Combinations of chemotherapeutic agents can be used.

In some embodiments, the chemotherapeutic agent is a topoisomerase inhibitor. In some instances, the topoisomerase inhibitor is a topoisomerase I inhibitor, a topoisomerase II inhibitor, or a combination thereof. In particular embodiments, the topoisomerase inhibitor is selected from the group consisting of: doxorubicin, etoposide, teniposide, daunorubicin, mitoxantrone, amsacrine, an ellipticine, aurintricarboxylic acid, HU-331, irinotecan, topotecan, camptothecin, lamellarin D, resveratrol, genistein, quercetin, epigallocatechin gallate (EGCG), and a combination thereof. EGCG is one example of a plant-derived natural phenol that serves as a suitable topoisomerase inhibitor. In some instances, the topoisomerase inhibitor is doxorubicin.

Immunotherapy refers to any treatment that uses the subject's immune system to fight a disease (e.g., cancer). Immunotherapy methods can be directed to either enhancing or suppressing immune function. In the context of cancer therapies, immunotherapy methods are typically directed to enhancing or activating immune function. In some instances, an immunotherapeutic agent comprises a monoclonal antibody that targets a particular type or part of a cancer cell. In some cases, the antibody is conjugated to a moiety such as a drug molecule or a radioactive substance. Antibodies can be derived from mouse, chimeric, or humanized, as non-limiting examples. Non-limiting examples of therapeutic monoclonal antibodies include alemtuzumab, bevacizumab, cetuximab, daratumumab, ipilimumab (MDX-101), nivolumab, ofatumumab, panitumumab, pembrolizumab, rituximab, tositumomab, and trastuzumab.

Immunotherapeutic agents can also comprise an immune checkpoint inhibitor, which modulates the ability of the immune system to distinguish between normal and “foreign” cells. Programmed cell death protein 1 (PD-1) and protein death ligand 1 (PD-L1) are common targets of immune checkpoint inhibitors, as disruption of the interaction between PD1 and PD-L1 enhance the activity of immune cells against foreign cells such as cancer cells. Examples of PD-1 inhibitors include pembrolizumab and nivolumab. An example of a PD-L1 inhibitor is atezolizumab. Another immune checkpoint target for the treatment of cancer is cytotoxic T lymphocyte-associated protein 4 (CTLA-4), which is a receptor that downregulates immune cell responses. Therefore, drugs that inhibit CTLA-4 can increase immune function. An example of such a drug is ipilimumab, which is a monoclonal antibody that binds to and inhibits CTLA-4.

Small molecule drugs generally are pharmacological agents that have a low molecular weight (i.e., less than about 900 Daltons). Non-limiting examples of small molecule drugs used to treat cancer include bortezomib (a proteasome inhibitor), imatinib (a tyrosine kinase inhibitor), and seliciclib (a cyclin-dependent kinase inhibitor), and epacadostat (an indoleamine 2,3-dioxygenase (IDO1) inhibitor).

The term “radiotherapy” refers to the delivery of high-energy radiation to a subject for the treatment of a disease (e.g., cancer). Radiotherapy can comprise the delivery of X-rays, gamma rays, and/or charged particles. Radiotherapy can be delivered locally (e.g. to the site or region of a tumor), or systemically (e.g., a radioactive substance such as radioactive iodine is administered systemically and travels to the site of the tumor).

The term “hormone therapy” can refer to an inhibitor of hormone synthesis, a hormone receptor antagonist, or a hormone supplement agent. Inhibitors of hormone synthesis include but are not limited to aromatase inhibitors and gonadotropin releasing hormone (GnRH) analogs. Hormone receptor antagonists include but are not limited to selective receptor antagonists and antiandrogen drugs. Hormone supplement agents include but are not limited to progestogens, androgens, estrogens, and somatostatin analogs. Aromatase inhibitors are used, for example, to treat breast cancer. Non-limiting examples include letrozole, anastrozole, and aminoglutethimide. GnRH analogs can be used, for example, to induce chemical castration. Selective estrogen receptor antagonists, which are commonly used for the treatment of breast cancer, include tamoxifen, raloxifene, toremifene, and fulvestrant. Antiandrogen drugs, which bind to and inhibit the androgen receptor, are commonly used to inhibit the growth and survival effects of testosterone on prostate cancer. Non-limiting examples include flutamide, apalutamide, and bicalutamide.

The term “differentiating agent” refers to any substance that promotes cell differentiation, which in the context of cancer can promote malignant cells to assume a less stem cell-like state. A non-limiting example of an anti-cancer differentiating agent is retinoic acid.

In the context of the embodiments of the present disclosure, the terms “treatment,” “treat,” “treating” and the related terms and expressions refer to reducing one or more of the effects (i.e., symptoms) of a disease or condition of a subject (e.g., one or more symptoms of a coronavirus infection or of cancer) by eliciting an immune response in the subject. Thus in the disclosed method, treatment can refer to a 10%, 20%, 30%, 40%, 50%, 60%, 70%, 80%, 90%, or 100% reduction in the severity of an established symptom or measurement of the disease or condition. For example, a method for treating a disease or condition is considered to be a treatment if there is a 10% reduction in one or more symptoms of the disease or condition in a subject, as compared to a control subject (e.g., an untreated subject). Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease or condition in the subject. Treating also refers to an action, for example, administration of a composition that occurs before or at about the same time a subject begins to show one or more symptoms of the condition or disease, which inhibits or delays onset or exacerbation or delays recurrence of one or more symptoms of the infection. As used in the present disclosure, references to decreasing, reducing, or inhibiting include a change of 10%, 20%, 30%, 40%, 50%, 60%, 70%, 80%, 90% or greater in the subject as compared to a control level. Thus, the reduction in onset, exacerbation or recurrence of the disease or condition can be about a 10, 20, 30, 40, 50, 60, 70, 80, 90, 100%, or any amount of reduction in between as compared to control subjects.

### EXEMPLARY EMBODIMENTS

Embodiment 1 is a composition comprising an isolated Staphylococcus aureus (S. aureus) peptidoglycan (PGN) sacculus bonded to an immunogenic polypeptide via a triazole moiety.

Embodiment 2 is the composition of Embodiment 1, wherein the triazole moiety is a reaction product between an azide-modified D-amino acid on the S. aureus PGN sacculus and a cycloalkyne moiety bonded to the immunogenic polypeptide via a linker moiety.

Embodiment 3 is the composition of Embodiment 2, wherein the azide-modified D-amino acid is azido-D-alanine (azaDala).

Embodiment 4 is the composition of Embodiments 2 or 3, wherein the linker moiety is a reaction product between an amino acid residue in the immunogenic polypeptide and a crosslinker reagent comprising the cycloalkyne moiety and a peptide-reactive handle.

Embodiment 5 is the composition of Embodiment 4, wherein the cycloalkyne moiety is a cyclooctyne moiety.

Embodiment 6 is the composition of Embodiment 5, wherein the cyclooctyne moiety is dibenzocyclooctyne (DBCO).

Embodiment 7 is the composition of any one of Embodiments 4-6, wherein the peptide-reactive handle is a maleimide and the amino acid residue in the immunogenic polypeptide is a cysteine residue.

Embodiment 8 is the composition of Embodiment 4, wherein the peptide-reactive handle is a N-hydroxysuccinimide moiety.

Embodiment 9 is the composition of any one of Embodiments 4, 7, or 8, wherein the crosslinker reagent further comprising one or more ethylene glycol moieties.

Embodiment 10 is the composition of Embodiment 9, wherein the one or more ethylene glycol moieties comprise polyethylene glycol (PEG).

Embodiment 11 is the composition of Embodiment 10, wherein the PEG is PEG3, PEG4, or PEG8.

Embodiment 12 is the composition of any one of Embodiments 1-11, wherein the S. aureus PGN sacculus is a peptidoglycan sacculus selected from S. aureus strain ATCC 25923, ATCC 29213, SH1000, RN4220, or RN4220 (ΔTarO).

Embodiment 13 is the composition of Embodiment 12, wherein the S. aureus PGN sacculus is a peptidoglycan sacculus from S. aureus strain SH1000.

Embodiment 14 is the composition of any one of Embodiments 1-13, wherein the S. aureus PGN sacculus is bonded to a plurality of different immunogenic polypeptides via triazole moieties.

Embodiment 15 is the composition of any one of Embodiments 1-14, wherein the immunogenic polypeptide is a viral protein, a bacterial protein, a fungal protein, or a protein expressed in a cancer cell.

Embodiment 16 is the composition of any one of Embodiments 1-15, wherein the immunogenic polypeptide is a SARS-CoV-2 Spike protein or a fragment thereof.

Embodiment 17 is the composition of any one of Embodiments 1-16, wherein the immunogenic polypeptide is a SARS-CoV-2 Spike protein receptor binding domain (RBD).

Embodiment 18 is the composition of any one of Embodiments 1-15, wherein the immunogenic polypeptide is a cancer neoantigen.

Embodiment 19 is the composition of any one of Embodiments 1-18, wherein the composition further comprises an adjuvant and/or a stabilizing agent.

Embodiment 20 is the composition of any one of Embodiments, wherein the stabilizing agent comprises nanoparticle hydrogel.

Embodiment 21 is the composition of Embodiment 20, wherein the adjuvant and/or the stabilizing agent are conjugated to the S. aureus PGN sacculus.

Embodiment 22 is the composition of any one of Embodiment) 1-21, wherein the adjuvant comprises at least one of a Toll-like receptor (TLR) agonist and/or a T-cell epitope.

Embodiment 23 is a formulation comprising the composition of any one of Embodiment(s)s 1-22 and a pharmaceutically acceptable excipient.

Embodiment 24 is the formulation of Embodiment 23, further comprising an adjuvant.

Embodiment 25 is a method of inducing an immune response in a subject, the method comprising administering to the subject a therapeutically effective amount of the formulation of Embodiment(s) 23 or 24.

Embodiment 26 is the method of Embodiment 25, wherein the method elicits an antibody response in the subject.

Embodiment 27 is the method of Embodiment 25, wherein the method elicits a T cell response in the subject.

Embodiment 28 is the method of any one of Embodiments 25-27, wherein the immunogenic polypeptide is a SARS-CoV-2 Spike protein or a fragment thereof, and wherein the formulation is administered in an amount capable of eliciting a protective immune response against the SARS-CoV-2 Spike protein in the subject.

Embodiment 29 is the method of Embodiment 28, wherein the subject has a SARS-CoV-2 infection, is suspected of having a SARS-CoV-2 infection, or is at risk of exposure to SARS-CoV-2 infection.

Embodiment 30 is the method of any one of Embodiments 25-27, wherein the immunogenic polypeptide is a protein expressed in a cancer cell, and wherein the formulation is administered in an amount capable of eliciting a protective immune response against a cancer.

Embodiment 31 is the method of Embodiment 30, wherein the protective immune response comprises production of neutralizing antibodies against the cancer in the subject.

Embodiment 32 is the method of Embodiment 30 or 31, wherein the subject has, has had, or is at risk of developing the cancer.

Embodiment 33 is the method of any one of Embodiments 25-32, wherein the composition is administered to the subject subcutaneously, intramuscularly, intravenously, intranasally, or orally.

Embodiment 34 is a kit comprising the formulation of Embodiment 23 or 24 packaged in a container and instructions for the administration thereof.

Embodiment 35 is the kit of Embodiment 34, further comprising an adjuvant.

Embodiment 36 is the kit of Embodiment 34 or 35, wherein the composition is lyophilized.

Embodiment 37 is the kit of any one of Embodiments 34-36, further comprising an applicator.

Embodiment 38 is the kit of any one of Embodiments 34-37, wherein the formulation is present in an effective amount, dosage unit, or plurality of dosage units.

Publications cited herein and the material for which they are cited are hereby specifically incorporated by reference in their entireties. The following description provides further non-limiting examples of the disclosed compositions and methods.

### Examples

The following examples are offered to illustrate, but not to limit the claimed disclosure. Methods and materials are provided below.

### Example 1. Modified PGN Sacculi Isolation

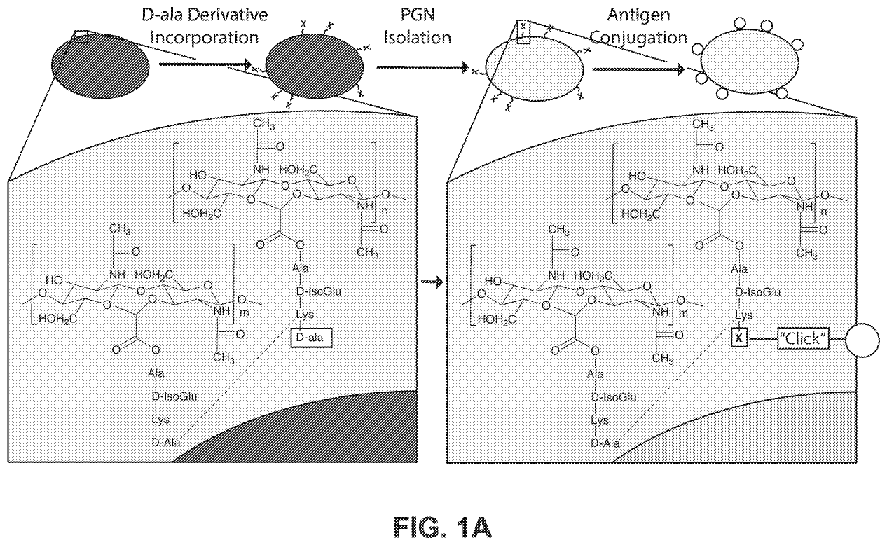

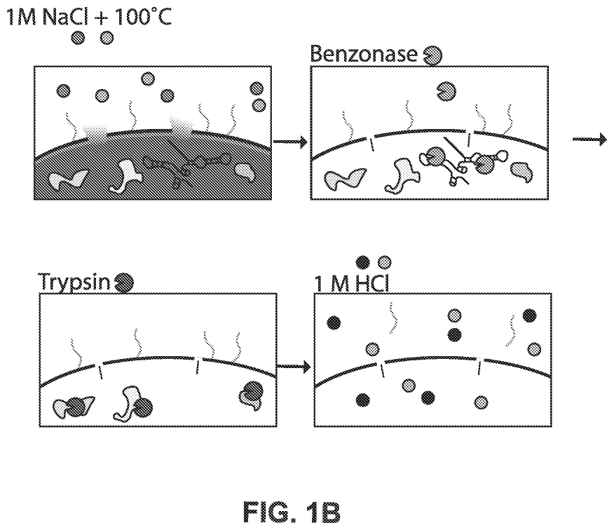

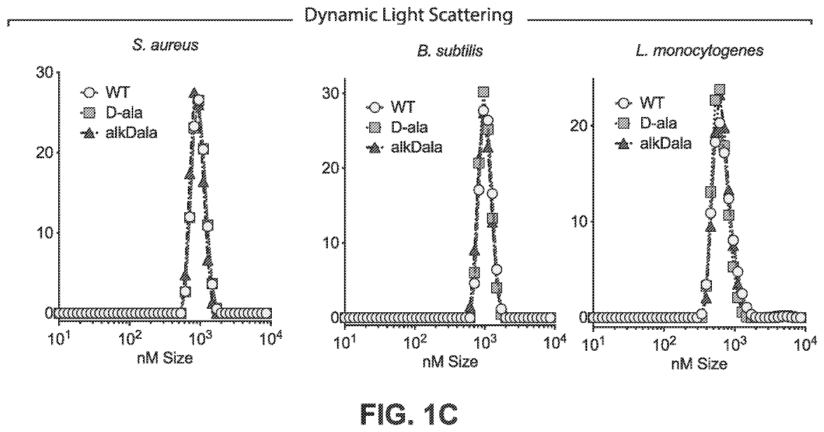

In order to produce a conjugatable bacterial PGN microparticle, the inventors first set out to develop and understand purified sacculi containing unnatural D-aa derivatives. The inventors set out to incorporate unnatural D-aa derivatives into the PGN shell of growing bacteria and then isolate the resultant microparticles (FIG. 1A; left panel) (Kuhner et al., 2014, “From Cells to Muropeptide Structures in 24 h: Peptidoglycan Mapping by UPLC-MS.” Sci. Rep. 4, 7494.doi.org/10.1038/srep07494; Zhou et al., 2012, “Nutrient-Dependent Structural Changes in S. aureus Peptidoglycan Revealed by Solid-State NMR Spectroscopy.” Biochemistry, 51(41), 8143-8153.doi.org/10.1021/bi3012115). Briefly, growing bacteria were incubated with a D-alanine (D-ala) derivative, which was incorporated into the terminal two positions of the stem peptides in their PGN (FIG. 1A; right panel) (Siegrist et al., 2013, “D-Amino Acid Chemical Reporters Reveal Peptidoglycan Dynamics of an Intracellular Pathogen.” ACS Chem. Biol. 8 (3), 500-505.doi.org/10.1021/cb3004995). The PGN shell of these bacteria was then isolated (FIG. 1B) via multiple harsh steps, including boiling in high osmolarity as well as an incubation in 1 M HCl. The inventors then set out to conjugate the purified PGN microparticles to an immunogen of interest by ligation to the chemical handle on the unnatural D-ala derivative (FIG. 1A).

### Example 2. Modified PGN Sacculi Characterization

To assess whether the incorporation of a modified D-ala residue impacted the PGN structure, the inventors used dynamic light scattering (DLS) to study the size of the purified sacculi from untreated cells and cells grown in the presence of either 1 mM D-ala or 1 mM alkynyl-D-ala (alkDala) (FIG. 1C). After purification, for the Gram-positive species, the microparticles showed no significant difference between any of the tested growth conditions: wildtype (WT), 1 mM D-ala, or 1 mM alkDala (FIG. 1C). This finding suggests that incorporation of an unnatural amino acid into the PGN shell did not impact the particle size or integrity of the five strains of Gram-positive bacteria tested. However, purified PGN microparticles of Gram-negative Escherichia coli showed large variations in size based on growth conditions. For instance, the incorporation of a modified D-ala residue reduced the size of the purified sacculi from E. coli, while the incorporation of a modified alkDala residue increased the size of the purified sacculi from E. coli (data not shown). These variations could be due to decreased stability of the thinner PGN shell in Gram-negative E. coli during PGN isolation compared to the Gram-positive strains. (Silhavy et al., 2010, “The Bacterial Cell Envelope.” Cold Spring Harb. Perspect. Biol. 2 (5), a000414-a000414. doi.org/10.1101/cshperspect.a000414). The inventors also examined genetically modified sacculi in PGN 0-acetyltransferase A (OatA) and N-acetylglucosamine deacetylase. (PgdA) mutants in Listeria monocytogenes. The PGN from both OatA- and PgdA-expressing cells was previously shown to modify the immune response and also to alter particle degradation by lysozyme-like enzymes. DLS measurements did not reveal any differences in size (data not shown).

### Example 3. Modified PGN Immunostimulatory Properties

Having demonstrated that the particle integrity of Gram-positive PGN shells remained intact after unnatural D-aa incorporation and purification, the inventors next sought to address the immunostimulatory properties of modified PGN microparticles in an NF-κB stimulation reporter cell line, RAW-Blue cells. (Lewis et al., 2014, “Macrophage Reporter Cell Assay for Screening Immunopharmacological Activity of Cell Wall-Active Antifungals.” Antimicrob. Agents Chemother. 58 (3), 1738-1743.doi.org/10.1128/AAC.02408-13.) RAW-Blue cells are immortalized murine macrophages that express relevant PGN immune receptors (TLR-2, NOD1, NOD2) and that produce secreted embryonic alkaline phosphatase upon activation. The inventors plated macrophages in the presence of PGN microparticles at a 10:33 ratio of cells to PGN and measured NF-κB activation with a colorimetric assay readout. The inventors aimed to ensure that unnatural D-aa incorporation did not influence immunostimulation and that the purified PGN microparticles remained immunostimulatory. The specific protocol is provided below.

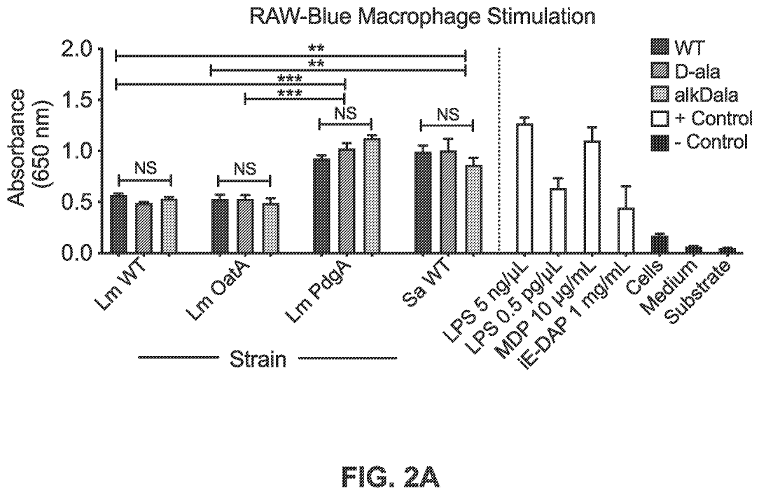

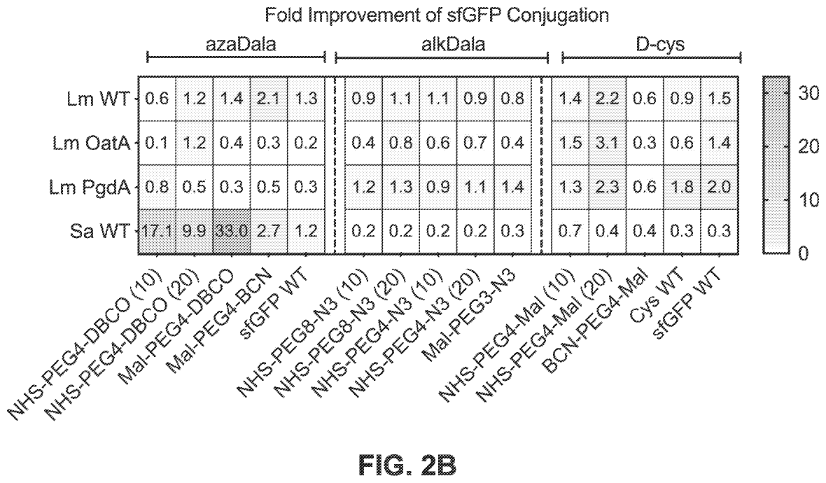

Wild type (WT) sacculi and those grown in the presence of additional D-alanine or of the unnatural amino acid alkDala displayed no significant difference in macrophage activation for the majority of the Gram-positive samples (FIG. 2A). However, E. coli, the only Gram-negative sample tested here, displayed differing responses (data not shown), perhaps due to heterogeneity in sizes or purity. For instance, E coli PGN microparticles with additional alkDala increased macrophage stimulation, while additional D-ala had no significant impact on macrophage stimulation (data not shown). On the contrary, Bacillus subtilis PGN microparticles with either D-ala or alkDala decreased macrophage stimulation (data not shown). Considering the DLS data discussed above, as well as this result, E. coli was not pursued in future experiments. Moreover, since Bacillus subtilis also exhibited markedly different macrophage activation between modifications of PGN microparticles (data not shown), it was not carried forward for further analysis.

The inventors found that the sacculi isolated from OatA-expressing cells were less immunogenic in the RAW-Blue assay than WT or PgdA-expressing L. monocytogenes strains (FIG. 2A) (Bera, et al., 2005, “Why Are Pathogenic Staphylococci so Lysozyme Resistant? The Peptidoglycan O-Acetyltransferase OatA Is the Major Determinant for Lysozyme Resistance of Staphylococcus Aureus.” Mol. Microbiol. 55 (3), 778-787.doi.org/10.1111/j.1365-2958.2004.04446.x). Taken together, these results demonstrate that Gram-positive bacterial PGN can yield homogeneous microparticles that are immunostimulatory.

### Example 4. Conjugation of Immunogenic Polypeptide to Modified PGN Sacculi

To evaluate the efficacy of the modified PGN scaffold as a vaccine microparticle, the inventors analyzed the ability to conjugate a subunit immunogen to purified sacculi. In order to facilitate comparisons, the inventors utilized the fluorescent, monomeric 26.8 kDa protein superfolder green fluorescent protein (sfGFP) as a model antigen. (Pédelacq, et al., 2006, “Engineering and Characterization of a Superfolder Green Fluorescent Protein. Nat. Biotechnol. 24 (1), 79-88.doi.org/10.1038/nbt1172; Gambotto et al., 2000. “Immunogenicity of Enhanced Green Fluorescent Protein (EGFP) in BALB/c Mice: Identification of an H2-Kd-Restricted CTL Epitope.” Gene Ther. 7(23), 2036-2040.doi.org/10.1038/sj.gt.3301335). The inventors tested the conjugation efficiency of sfGFP to the four Gram-positive sacculi (S. aureus and three derivatives of L. monocytogenes) with three D-aa derivatives incorporated into their sacculi. The inventors purified PGN microparticles containing azaDala, alkDala, or D-cys from these four strains. The inventors then tested the conjugation efficiency of 14 derivatives of sfGFP modified in various ways with the corresponding clickable handles. (Sletten and Bertozzi, 2009. “Bioorthogonal Chemistry: Fishing for Selectivity in a Sea of Functionality.” Angew. Chemie-Int. Ed. 48 (38), 6974-6998.doi.org/10.1002/anie.200900942). sfGFP was modified with strained cyclooctynes, azides, or maleimides through n-hydroxysuccinimide (NHS) or maleimide chemistries (FIG. 2B). The inventors analyzed the conjugation efficiency of these click-reactions by flow cytometry, comparing the mean fluorescence intensity (MFI) of the PGN shells with or without the corresponding D-aa incorporation (FIG. 2B). The specific protocols used are described in detail below.

S. aureus PGN modified with azaDala (henceforth referred to as “SA Aza-Pep”), affords the highest conjugation efficiency when conjugated to sfGFP derivatives containing dibenzocyclooctyne (DBCO) functionalities (FIG. 2B, left). The site-specific maleimide-Peg4-DBCO conjugation to a cysteine at the 3-position on sfGFP yielded more effective conjugation than the similar NHS-PEG4-DBCO conjugations (FIG. 2B). The resulting maleimide-PEG4-DBCO conjugation of SA Aza-Pep to sfGFP produced an immunogen (henceforth referred to as “sfGFP-SA-Aza-Pep”). No modification was detected for any condition for the three derivatives of L. monocytogenes (FIG. 2B) which was surprising because these bacteria readily incorporate D-aa into their PGN. (Siegrist et al., 2013).

### Example 5. Immunization with Modified PGN Sacculi Conjugated to sfGFP

Given the successful conjugation of a subunit vaccine candidate (sfGFP) to the surface of a S. aureus PGN microparticle, the inventors investigated this microparticle's in vivo efficacy as an immunogen. The inventors sought to compare the immunogenicity of PGN microparticles to KLH conjugates with and without Freund's adjuvant system (Freund's complete adjuvant for the prime, followed by Freund's incomplete for the boosts) (Stills, 2005. “Adjuvants and Antibody Production: Dispelling the Myths Associated with Freund's Complete and Other Adjuvants.” ILAR J. 46 (3), 280-293.doi.org/10.1093/ilar.46.3.280). Freund's adjuvant system is an extremely robust adjuvant that is not approved for any human or veterinary vaccines, but is only used in laboratory settings. The inventors produced a KLH-GFP conjugate using a maleimide-activated KLH in order to also conjugate sfGFP through the cysteine at the 3-position, and normalized the amount of sfGFP in each immunization sample using a standard sfGFP curve (data no shown). Therefore, each immunization sample had an identical amount of fluorescence. Moreover, given that the inventors were able to determine the molarity of sfGFP in each sample, the inventors approximated the number of sfGFP units per KLH (˜21 sfGFP/KLH) or per PGN microparticle (˜250,000 sfGFP/sacculi); by knowing the concentration of KLH used in the conjugation or by counting the number of microparticles in a known sample volume by microscopy.

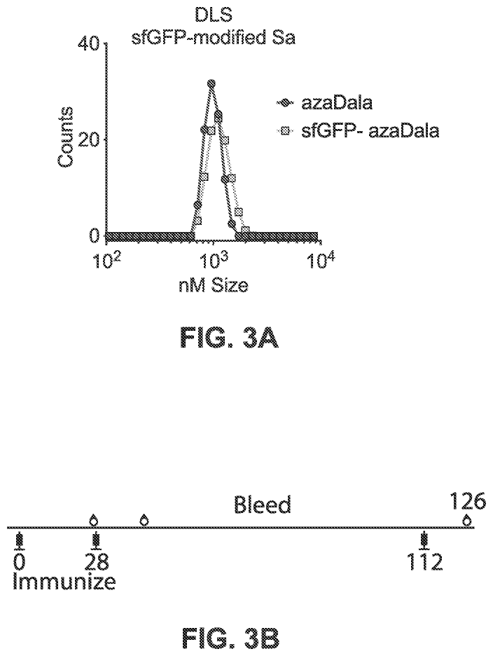

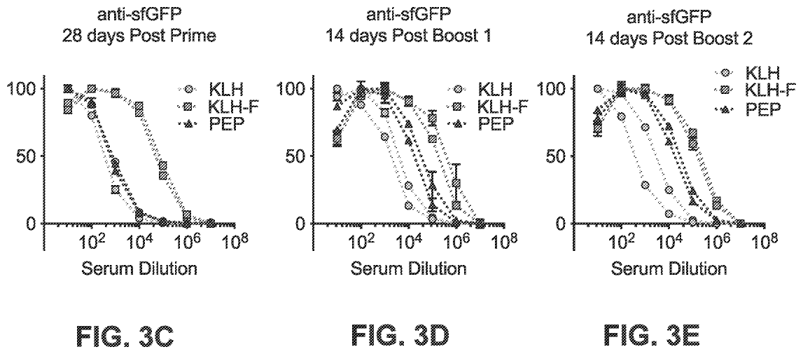

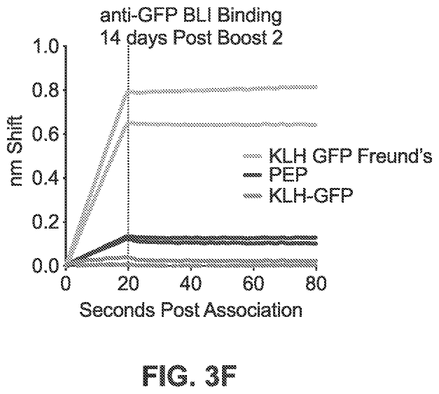

Prior to immunization the inventors ensured that the particles retained their integrity via DLS as shown in Example 1, which showed a minor increase in size due to the conjugation (FIG. 3A). Furthermore, given that serum contains lysozyme-like enzymes, the inventors aimed to understand the serum stability of these PGN conjugates. The inventors therefore employed flow cytometry to understand decreases in both sfGFP fluorescence due to serum proteases and particle degradation due to serum indicated lysozymes. In guinea pig serum the GFP component of SA-Aza-Pep had a half-life of 4.5 h compared to the 17.7 h for the PGN microparticles (data not shown). In rabbit serum the half-life was 2.4 h and 10.4 h, respectively (data not shown).

These analyses provided the groundwork for an immunization experiment in guinea pigs with 2.5 μg of sfGFP conjugated to KLH or as sfGFP-SA-Aza-Pep. The immunization protocol contained one boost at week 4 and a second boost at day 112 to investigate long-lived immunity (FIG. 3B). The inventors compared KLH conjugations with and without Freund's adjuvant to the adjuvant-free immunization of sfGFP-SA-Aza-Pep. The inventors profiled the immune response after the prime (FIG. 3C), first boost (FIG. 3D), and second boost (FIG. 3E). Following a boost, the unadjuvanted sfGFP-SA-Aza-Pep elicited a better immune response than unadjuvanted KLH; however, this response was less robust than with the Freund's adjuvant system (FIGS. 3C-D). Strikingly, following a boost at day 112, sfGFP-SA-Aza-Pep retained a robust response (FIG. 3E), demonstrating PGN's ability to elicit an anamnestic immune response. Further, biolayer interferometry, where the magnitude of the nm shift can be used as a surrogate for binding, indicated the same rank order of binding, with PGN microparticles out-performing KLH (FIG. 3F).

### Example 6. Immunogenicity of Various S. aureus Modified PGN Sacculi Conjugated to sfGFP

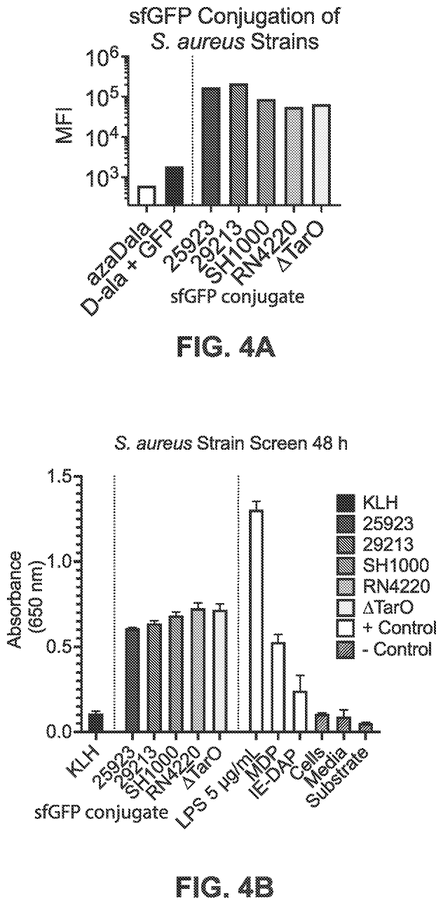

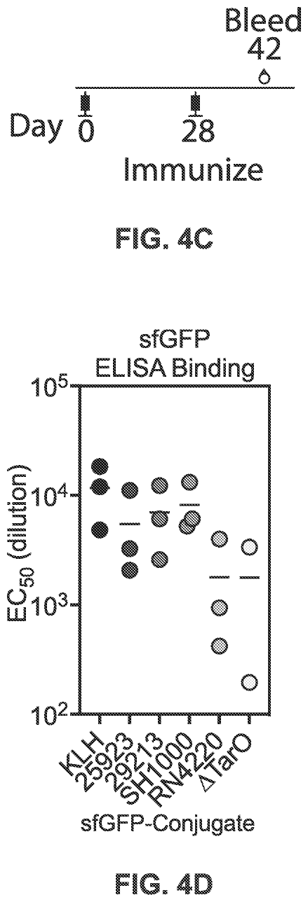

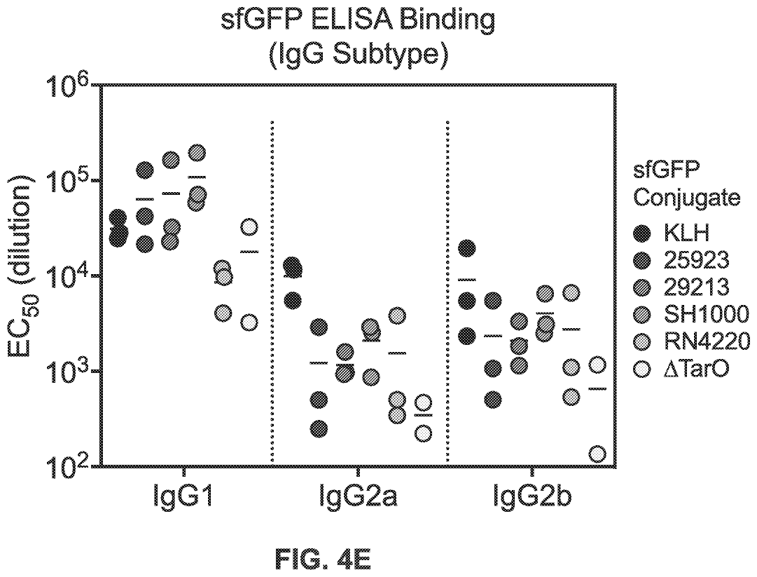

Next, the inventors investigated whether the S. aureus strains used to produce the PGN microparticles played a role in immune response. The inventors conducted an experiment testing five sfGFP-conjugated S. aureus strains for their immunogenicity in mice. The inventors selected two common (Rosenbach) lab strains (ATCC: 25923 and 29213), two other common laboratory strains (SH1000 and RN4220), and a wall teichoic acid knockout of RN4220 (ΔTarO) for the experiment. (Rosenbach, A. J. F. Mikro-Organismen Bei Den Wund-Infections-Krankheiten Des Menschen; J. F. Bergmann, 1884; Vol. 2; Horsburgh, et al., 2002. “SigmaB Modulates Virulence Determinant Expression and Stress Resistance: Characterization of a Functional RsbU Strain Derived from Staphylococcus Aureus 8325-4.” J. Bacteriol. 184 (19), 5457-5467. doi.org/10.1128/JB.184.19.5457-5467.2002; Kreiswirth et al., 1983. “The Toxic Shock Syndrome Exotoxin Structural Gene Is Not Detectably Transmitted by a Prophage.” Nature. 305 (5936), 709-712.doi.org/10.1038/305709a0; Wang et al., 2013. “Discovery of Wall Teichoic Acid Inhibitors as Potential Anti-MRSA β-Lactam Combination Agents.” Chem. Biol. 20 (2), 272-284.doi.org/10.1016/j.chembiol.2012.11.013). Sacculi were isolated from all five strains and conjugated using maleimide-PEG4-DBCO modified sfGFP. Using flow cytometry, the inventors determined the relative conjugation efficiency to be similar across all strains tested (FIG. 4A). The inventors also confirmed that all strains of PGN yielded similar macrophage stimulation with RAW-Blue assay, when normalizing for amount of sfGFP (FIG. 4B). Differences in silver staining (data not shown) suggested that bacterial components (such as wall teichoic acids, for example) may impact PGN purification efficiency during the purification protocol. For instance, the strain RN4220 shows the most contaminants, smearing of dark colors by silver stain, followed by the strains ATCC. 29213, SH1000, ATCC: 25293 and ΔTarO (data not shown).

All five PGN strains and KLH were conjugated with sfGFP and then injected into three mice each, with the exception of ΔTarO which was immunized into two mice. Mice were immunized at day 0 and boosted at day 28; ELISAs were conducted with serum collected 14 days post boost (FIG. 4C). The immune response was strikingly similar between KLH and PGN as mice elicited a more robust sfGFP-KLH response and slightly weaker PGN response than guinea pigs (FIGS. 3D and 4D). However, not all S. aureus strains elicited a response similar to KLH: notably, two strains, RN4220 and the ΔTarO derivative of RN4220, elicited a weaker overall response relative to the other strains (FIG. 4D).

The inventors then profiled the IgG subtypes involved in the response in order to profile overall Th2- or Th1-type responses, known as immune polarization. Adjuvants typically alter immune polarization and bacterial infections typically promote a Th2-type response (Pulendran et al., 2021. “Emerging Concepts in the Science of Vaccine Adjuvants.” Nat. Rev. Drug Discov. 20 (6), 454-475.doi.org/10.1038/s41573-021-00163-y; D'Elios et al., 2011. “T-Cell Response to Bacterial Agents.” J. Infect. Dev. Ctries. 5 (9), 640-645. doi.org/10.3855/jidc.2019). This polarization can be profiled by analyzing the relative ratios of IgG1/IgG2 in mice, where IgG1-skewed responses favor Th2-type responses. Given that bacterial infections promote a Th2-type response, it is not surprising that all five sfGFP-PGN conjugate vaccines elicited a preferentially IgG1 response versus IgG2a/b responses (FIG. 4E). Other common adjuvants, for example alum, also promote a Th2-type response. The sfGFP-SH1000 conjugate vaccine appeared to produce the most robust response among the PGN microparticles that were assessed (FIG. 4D) as well as a ratio of IgG1 to IgG2a/b that was more even between IgG1 and IgG2a/b compared to other strains tested (FIG. 4E). For this reason, SH1000 PGN was selected for further investigation.

### Example 6. Modified PGN Sacculi Conjugated to SARS-CoV-2 RBD

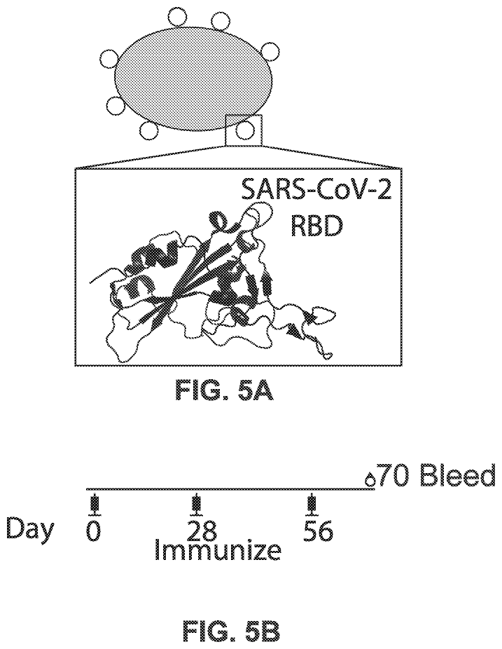

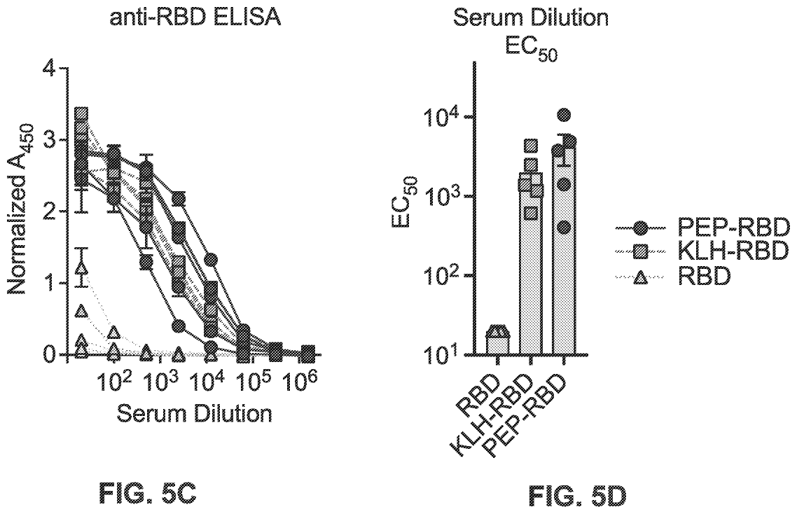

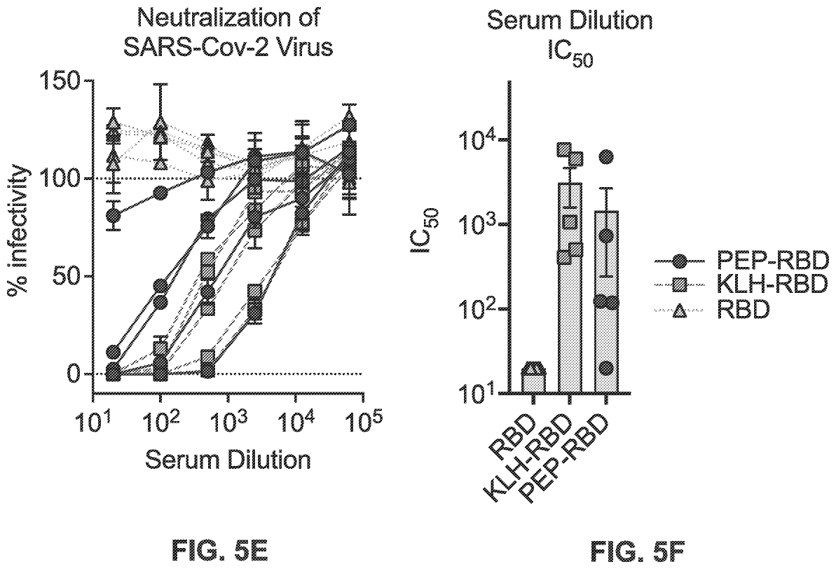

To test whether PGN was a suitable vaccine microparticle for a viral immunogen, the inventors turned to the SARS-CoV-2 receptor binding domain (RBD) (FIG. 5A). The RBD has a molecular weight similar to that of sfGFP and was previously shown to produce a skewed IgG1 type response in mice. (Amanat, et al., 2020. “A Serological Assay to Detect SARS-CoV-2 Seroconversion in Humans.” Nat. Med. 26 (7), 1033-1036.doi.org/10.1038/s41591-020-0913-5; Powell et al., 2021. “A Single Immunization with Spike-Functionalized Ferritin Vaccines Elicits Neutralizing Antibody Responses against SARS-CoV-2 in Mice.” ACS Cent. Sci. 7(1), 183-199). The inventors conjugated the RBD to PGN microparticles through maleimide-PEG4-DBCO conjugated to Cys538 (spike numbering) (FIG. 5A). Although the Cys538 position should be a free cystine residue in the RBD construct, previous reports indicated that Cys538 is spontaneously cysteinylated. (Ramirez et al., 2021. “SARS-CoV-2 RBD-Tetanus Toxoid Conjugate Vaccine Induces a Strong Neutralizing Immunity in Preclinical Studies.” ACS Chem. Biol. 16 (7), 1223-1233.doi.org/10.1021/acschembio.1c00272). Therefore, mild tris(2-carboxyethyl)phosphine (TCEP) reduction was utilized to facilitate DBCO conjugation. Modification was confirmed using the absorbance of DBCO at 310 nm. For instance, without reduction there was no reaction of SARS-CoV-2 RBD with the maleimide (data not shown).

Biolayer interferometry confirmed that mildly reduced and DBCO-modified SARS-CoV-2 RBD still bound to conformation-specific antibodies and ACE2. In particular, biolayer interferometry measurements demonstrates binding of DBCO-modified SARS-CoV-2 RBD to three conformation-specific antibodies (e.g., CR3022 (SEQ ID NOs: 5 and 6), CB6 (SEQ ID NOs: 7 and 8), CoVA2-15 (SEQ ID NOs: 9 and 10) or hFc-ACE2 (SEQ ID NO:11) is similar comparable to the control unreduced SARS-CoV-2 RBD. This data shows reduction and modification did not significantly impact binding (data not shown).

### Example 7. Immunization with Modified PGN Sacculi Conjugated to SARS-CoV-2 RBD

Finally, the inventors conjugated the reduced RBD to KLH and the DBCO-modified RBD to SH1000 sacculi and normalized the amount of RBD in each conjugate. RBD is not fluorescent; hence, the inventors were unable to use a fluorescence-based standard curve. Instead, the inventors utilized an anti-His tag dot blot standard curve to bridge the gap between a known concentration of His-tagged sfGFP-modified KLH or PGN microparticles (determined using fluorescence) and His-tagged RBD-modified KLH or PGN microparticles (data not shown).

The inventors then immunized mice with WT RBD, KLH-RBD, or SH1000-RBD in phosphate-buffered saline with no other adjuvants, normalized for the amount of RBD. The vaccination schedule is shown in FIG. 5B. FIG. 5C shows ELISA binding of serum from mice immunized with SARS-CoV-2-RBD-conjugates, FIG. 5D shows EC₅₀ derived from the curves in FIG. SC, FIG. 5E shows neutralization curves of heat-inactivated serum from mice immunized with SARS-CoV-2-RBD-conjugates, and FIG. 5F shows IC₅₀ derived from the curves in FIG. 5E (mean and SEM). The results show that following a prime and two boosts (FIG. 5B), the unconjugated RBD elicited a weak response, with only two animals showing any indication of binding and no indication of neutralization (FIGS. 5C-5F). In contrast, both the KLH- and SH1000-conjugated RBDs displayed significant binding and potent neutralization (FIGS. SC-5F). As with the sfGFP-immunized mice, there was little difference between the KLH- and SH1000-conjugated groups in terms of neutralization (FIGS. 5C-5D).

Taken together, these results demonstrate that PGN microparticles can serve as highly conjugatable, immunostimulatory, biodegradable microparticles with comparable immunoactivation as the commonly used carrier protein KLH. Given their ease of purification and scale up, PGN microparticles represent a novel and adaptable vaccine platform.

### Example 8. Materials and Methods

The following materials and methods were used in performing the experiments described in Examples 1-7.

Peptidoglycan (PGN) Production and Isolation. Wild type (WT), OatA- or PgdA-expressing bacteria were grown to saturation in LB medium. This suspension was diluted 1:10,000 into 3-4 mL LB containing 1 mM of the unnatural D-ala derivative and grown overnight to saturation. Cultures were harvested by centrifugation at 13,000×g for 1 min and washed twice with 1× phosphate-buffered saline (PBS). For isolation, cells were resuspended in 1 M NaCl (for Gram-positive cells) or 0.1 M Tris/HCl pH 7+0.25% SDS (for Gram-negative cells) and boiled for 30 min at 100° C. These suspensions were washed twice with ddH₂O, resuspended in 500 μL ddH₂O, and sonicated in a water bath for 30 min. Five hundred microliters of Tris buffer pH 7.4 with 1 μL benzonanse (Sigma) were added to each sample and incubated at 37° C. for 1 h. Thirty microliters of trypsin (HyClone 0.25%) were added to each sample and incubated at 37° C. for 1 h. Samples were then boiled at 100° C. for 5 min and washed twice with ddH₂O. Finally, samples were incubated in 1 mL of 1 M HCl for 4 h at 37° C., washed with ddH₂O until their pH was approximately neutral, and then spun down at 13,000×g and resuspended into 1×PBS for use in conjugation reactions.

Dynamic Light Scattering (DLS) Measurements. DLS measurements were taken on a Malvern Zetasizer Nano-S. One hundred microliters of PGN suspension were added to a 70 μL disposable cuvette and the temperature was equilibrated to 25° C. for at least 2 min prior to sample measurement.

RAW-Blue NF-kB Activity Assay Protocol. RAW-Blue cells were purchased from InvivoGen and the macrophages were maintained as described by the manufacturer. Briefly, cells were maintained in DMEM medium supplemented with 4.5 g/L glucose, 10% fetal bovine serum (FBS), and 100 μg/mL Normocin™ (InvivoGen) and Zeocin® (InvivoGen). One hundred eighty microliters of cells were plated at 550,000 cells/mL in a 96-well dish with 20 μL of PGN, suspended in endotoxin-free H₂O. The amount of PGN per sample was previously quantified via serial dilution and imaged via confocal microscopy to achieve 3.3 PGN per macrophage in each well. For testing the Staphylococcus aureus strains, 3 μL of sample (10 μg/mL sfGFP) were used. The assays were conducted in the same medium except the FBS used in the assay was heat inactivated. Plates were incubated for 48 h at 37° C. and 5% CO₂. A 50 μL sample of the supernatant was taken and added to 150 μL of QUANTI-Blue (InvivoGen). The mixture was then incubated in 96-well flat-bottom plates for 30 min at 37° C. before quantification with a spectrophotometer (BioTek Synergy™ HT Microplate Reader (BioTek) at 650 nm). Experiments were conducted in triplicate of triplicate. Mean and standard deviation of the mean from the three experiments is shown. Data analysis was done on Graphpad Prism (v9) using an unpaired t test.

GFP Expression. The gene encoding sfGFP (SEQ ID NO: 3) was cloned into a pET28b vector with a C-terminal hexa-His tag. For all constructs in which a maleimide was conjugated or a free cysteine was utilized, a Cys residue was incorporated at position 3 (SEQ ID NO:4). The sfGFP-N3 construct containing a genetically encoded azido-phenyl alanine (pET22b-T5-sfGFP* and pUltra-Poly) were generously supplied by Professor Peter Schultz at Scripps. All cells were grown in 2XYT medium and induced at OD 0.6-0.8. In the case of sfGFP-N₃, 2XYT was supplemented with 1 mM azido-phenyl alanine (Chem Impex) dissolved in H₂O, solubilized dropwise with NaOH (conc.), and filtered with a 0.22-μm filter. Cells were induced with 1 mM IPTG and protein was expressed for 4 h at 37° C. with shaking.

GFP Purification. Escherichia coli Cells were harvested by centrifugation for 10 min at 5000×g and lysed with sonication. Sonicated samples were spun again at 13,000×g for 1 h and GFP was then purified from cell lysates through NiNTA purification (HisPur). sfGFP used for conjugation experiments was buffer exchanged into PBS. That used for all other experiments was run over endotoxin removal resin (Pierce) and FPLC purified (Superdex 200).

GFP Conjugation Evaluation. sfGFP conjugated samples were analyzed by flow cytometry (BD Accuri C6) and the mean fluorescence intensity (MFI) of the conjugated samples was divided by the unconjugated samples (exact sample conditions as above, but PGN lacked the clickable handle). Data shown in FIGS. 2A-2B. Samples compared were clicked (Aza+DBCO) to unclicked (WT+DBCO) (data not shown). The values calculated in FIG. 2B were found by dividing the MEI of the Aza+DBCO trace by that of the WT+DBCO trace. Data were analyzed using FlowJo software (available from www.flowjo.com).

Screening sfGFP Conjugation Conditions to Isolated PGN. Previously prepared sfGFP was conjugated through maleimide (two molar equivalents in 1×PBS), through N-hydroxysuccinimide (respective number of molar equivalents shown above in 1×PBS), or left unconjugated. After conjugation all samples were buffer exchanged into 1×PBS and diluted to 1 mg/mL. PGN isolated as described above was spun down at 13,000×g and resuspended in 1 mg/mL solutions of sfGFP derivatives with their respective clickable handles. Copper-free (Cu-free) click reactions and thiol-reactive samples were incubated for 1 h at room temperature. To the Cu click samples of a preformed complex of BTTAA (33 mM, two molar equivalents) and CuSO₄ (16.5 μM, 1 equivalent) was added freshly prepared 100 mM sodium ascorbate. Reactions were incubated at room temperature and then spun down and washed six times with PBS.

sfGFP Conjugation to PGN Microparticles and KLH. Samples were prepared as above using DBCO-conjugated sfGFP incubated at 12 mg/mL with S. aureus PGN (ATCC 25923 or other strains as described) (from 4 mL culture) containing an azido-D-ala. PGN samples were incubated and rotated for 96 h at room temperature and purified by spinning down and resuspending in PBS six times. Alternatively, Cys-3-GFP was mixed 1:1 each 10 mg/mL with maleimide KLH (Imject). KLH samples were prepared per the manufacturer's recommendation (2 h in 1×PBS). KLH was purified away from GFP using successive passes through a 100 kDa concentrator (Amicon) until no unconjugated GFP was seen in solution. Quantification of GFP concentration in each sample was done using a standard curve of GFP fluorescence (GraphPad Prism).

Guinea Pig Immunizations. Male guinea pigs were given intramuscular immunizations containing 2.5 μg of GFP in 100 μL of each sample (Josman LLC). Prior to immunizations, samples were mixed 1:1 with 1×PBS (PGN and KLH without Freund's samples) or 1:1 with Freund's complete (primary) and Freund's incomplete (subsequent immunizations) for KLH-GFP Freund's samples. Immunizations occurred on Day 0, 28, and 112, and bleeds were conducted on Day 0, 28, 42, and 126.

Guinea Pig and Rabbit Serum Stability Assays. Samples of sfGFP-conjugated PGN microparticles (ATCC: 25923) were incubated in 25% guinea pig or rabbit serum and 75% RPMI with shaking at 37° C. for 62 h. At 0, 1, 10, 24, 38, 48, and 62 h, 100 μL of samples were taken and flash frozen using liquid nitrogen. Following isolation of the final timepoint, samples were thawed simultaneously, added to a v-bottom plate, and analyzed by flow cytometry (BD Accuri C6). Gates were drawn to encompass PGN microparticles and the MFI and raw counts were collected. Curves were fit using a one-phase decay on GraphPad Prism 8.4.1.

Serum ELISAs. ELISAs were done essentially as previously described. (Weidenbacher, et al., 2019, “Protect, Modify, Deprotect (PMD): A Strategy for Creating Vaccines to Elicit Antibodies Targeting a Specific Epitope.” Proc. Natl. Acad. Sci., 116 (20), 9947 LP—9952.doi.org/10.1073/pnas.1822062116). Briefly, plates (Maxisorb) were coated in 50 μL of 5 μg/mL sfGFP or RBD for 2 h at room temperature. Plates were washed three times with 1×PBST and then blocked with 1×PBST with 0.5% BSA (GFP) or ChonBlock (RBD) for at least 1 h at room temperature. Plates were washed once with 1×PBST, 50 μL of serial dilutions of guinea pig or mouse serum in 1×PBST were added to the plate for 1 h at room temperature, and the plates were washed three times with 1×PBST. An anti-guinea pig horse radish peroxidase secondary antibody (Abcam) at a 1:10,000 dilution or anti-mouse or anti-mouse IgG1/IgG2a/IgG2b (Abcam) in 1×PBST was added to each well and incubated for 1 h at room temperature. Finally, plates were washed four times with 1×PBST and 50 μL of 1-Step Turbo TMB-ELISA Substrate Solution (Thermo Fisher Scientific) were added to each well. Plates were quenched with 50 μL of 2 M H₂SO₄ and read on a spectrophotometer. Data were visualized using Graph Pad Prism 8.4.1.

RBD Purification. RBD was purified using HisPur™ Ni-NTA resin (ThermoFisher). Expi293F Cell supernatants were diluted with ⅓ volume wash buffer (20 mM imidazole, 20 mM HEPES pH 7.4, 150 mM NaCl) and the Ni-NTA resin was added to diluted cell supernatants. RBD was then incubated at 4° C. while stirring overnight. Resin/supernatant mixtures were added to chromatography columns for gravity flow purification. The resin in the column was washed with wash buffer (20 mM imidazole, 20 mM HEPES pH 7.4, 150 mM NaCl) and the RBD was eluted with 250 mM imidazole, 20 mM HEPES pH 7.4, 105 mM NaCl. Column elutions were concentrated using centrifugal concentrators (10 kDa cutoff for RBD), followed by size-exclusion chromatography on an AKTA Pure system (Cytiva). AKTA Pure FPLC with a Superose™ 6 Increase gel filtration column (S6) was used for purification. One milliliter of sample was injected using a 2 mL loop and run over the S6, which had been preequilibrated in degassed 20 mM HEPES, 150 mM NaCl prior to use. Biotinylated antigens were not purified using the AKTA.

RBD Conjugation to Isolated PGN. Previously prepared SARS-CoV-2 RBD conjugated with maleimide-PEG4-DBCO was mixed at 2 mg/mL to SH100 PGN microparticles (PGN microparticles isolated from 4 mL of SH1000 S. aureus growth). Prior to conjugation, RBD was sterile filtered using a 0.22-μm filter. SH1000 PGN microparticles were boiled at 100° C. for 20 min to sterilize them, as they could not be filtered due to their size. These mixed reactions were rocked at room temperature for three days and then spun down (13,000×g) and resuspended six times with PBS.

Quantification of RBD Conjugation. To quantify the amount of RBD in solution, the inventors developed a standard curve dot-blot using His-tagged sfGFP and RBD. sfGFP-conjugated KLH or PGN microparticles (of known sfGFP concentration, as determined with a standard curve) were dotted in duplicate on a nitrocellulose membrane (1.8 μL dots, ThermoFisher) in two-fold dilutions. Unknown concentrations of RBD-conjugated KLH or PGN microparticles were also dotted in duplicate. In a final lane, unmodified PGN microparticles were dotted as a control. An anti-his dot-blot was conducted as follows. The blot was dried for 15 min in a fume hood. Following drying, 10 mL of 1×PBST+5% blotting grade blocker (Bio-Rad) were added for 10 min. Two microliters of mouse anti-hexa His antibody (BioLegend) were added to the 10 mL sample and incubated for 1 h at room temperature. Blots were washed 16 times with 9 mL of PBST. Ten milliliters of 1×PBST+5% blotting grade blocker with 2 μL anti-mouse IgG1 (Abcam) were added and incubated for 1 h at room temperature. Blots were washed 16 times with 9 mL of PBST, developed using Pierce ECL Western blotting substrate, and imaged using a GE Amersham imager 600. Dots were quantified using the gel analysis protocol in Fiji (Version 1.0, ImageJ), and curves were fitted and unknown concentrations were evaluated using a linear regression in GraphPad Prism 8.4.1.

RBD Reduction Reaction and Conjugation to Mal-DBCO. Previously prepared SARS-CoV-2 RBD was reduced by the addition of one molar equivalent TCEP and left to incubate at room temperature for 1 h. Samples were then buffer exchanged into PBS using a PD-10 column (Cytiva). Following reduction, RBD was mixed with two molar equivalents of maleimide-PEG4-DBCO and left to react for 2 h at room temperature. Again, a PD-10 column was used to isolate reacted RBD from unreacted maleimide.

RBD and Monoclonal Antibody Expression. RBD, monoclonal antibodies, and soluble human ACE2-Fc (SEQ ID NO:11) were expressed and purified from Expi293F cells (ThermoFisher). Expi293F cells were cultured in 66% Freestyle/33% Expi medium (ThermoFisher) and grown in TriForest polycarbonate shaking flasks at 37° C. in 8% CO₂. One day prior to transfection, cells were spun down at 300×g and resuspended to a density of 3×10⁶ cells/mL in fresh medium. The next day, cells were diluted and transfected at a density of approximately 3-4×10⁶ cells/mL. Transfection mixtures were made by adding maxi-prepped DNA, culture medium, and FectoPro® transfection reagent (Polyplus) to cells to a ratio of 0.5-0.8 μg:100 μL:1.3 μL:900 μL. For example, for a 100-mL transfection, 50-80 μg of DNA were added to 10 mL of culture medium and then 130 μL of FectoPro® transfection reagent was added to that mixture. Following mixing and a 10 min incubation, the resultant transfection cocktail was added to 90 mL of cells. The cells were harvested 3-5 days after transfection by spinning the cultures at >7,000×g for 15 min. Supernatants were filtered using a 0.22-μm filter.

Protein Purification (Fc Tag). Anti-RBD IgGs and hFc-Ace2 fusions were purified using a 5 mL MAb Select Sure PRISM™ column on the AKTAT™ Pure FPLC (Cytiva). Filtered cell supernatants were diluted with 1/10 volume 10× Phosphate Buffered Saline (PBS). The AKTA™ system was equilibrated with 1×PBS for A1, 100 mM glycine pH 2.8 for A2, 0.5 M NaOH for B1, 1×PBS for the buffer line, and H₂O for the sample lines. The protocol involved washing the column with A1, then loading the sample in Sample line 1 until air was detected in the air sensor of the sample pumps, followed by five column volume washes with A1, elution of the sample by flowing of 20 mL of A2 (directly into a 50 mL conical containing 2 mL of 1 M Tris pH 8.0) followed by five column volumes of A1, B1, A1. The resultant Fc-containing samples were concentrated using 50 or 100 kDa cutoff centrifugal concentrators. Proteins were buffer exchanged using a PD-10 column (SEPHADEX) that had been preequilibrated into 20 mM HEPES, 150 mM NaCl. IgG-ACE2 fusions were further purified using the S6 column on the AKTA as above.

Biolayer Interferometry. Guinea pig serum was analyzed using an Octet Red 96. Serum was directly diluted into 1:100 into PBST+bovine serum albumin (BSA). Streptavidin biosensors were loaded with 100 nM biotinylated sfGFP which had been biotinylated using maleimide-PEG11-biotin (EZ-Link) as described above. Sensor tips were baselined in a 1:100 dilution of pre-immune serum, left to associate in 1:100 post-immune serum, and then dissociate back in the pre-immune serum. Plots were made using Graph-Pad Prism 8.4.1.

Mouse Immunizations. Samples of sfGFP-PGN microparticles from a variety of species and SARS-CoV-2 RBD-PGN microparticles from SH1000 or comparable KLH controls were prepared in 1×PBS at a concentration of 10 μg/mL of sfGFP or SARS-CoV-2 RBD. Quantification of GFP concentration in each sample was done using a standard curve of GFP fluorescence; for RBD, quantification was done using a standard curve produced by dot-blot (described above). One microgram of GFP or RBD (in 100 μL) of each sample was immunized intramuscularly into BALB/C mice (Jackson Laboratory). Immunizations and bleeds occurred following the schedules described in the figures.

Mutanolysin Digestion and Silver Stain Analysis. Ten microliters of 10 μg/mL sfGFP-conjugated PGN microparticles in 1×PBS were digested by the addition of 10 ng of mutanolysin followed by incubation overnight with shaking at 37° C. Following digestion, a silver stain was conducted (Pierce, ThermoFisher). The gel was run in clean running buffer (Bio-Rad), washed five times in ddH₂O, fixed with 30% ethanol: 10% acetic acid solution for 15 min, and replaced for another 15 min. The gel was then washed with 10% ethanol and ddH₂O twice for 5 min. Sensitizer working solution was made (one part Silver Stain Sensitizer and 500 parts ultrapure water) and incubated with the gel for 1 min. The gel was then washed twice for 1 min with ddH₂O. Next, the gel was incubated for 30 min in Stain Working Solution (one part Silver Stain Enhancer with 50 parts Silver Stain). The gel was washed twice in ddH₂O and then added to Developer Working Solution (one part Silver Stain Enhancer with 50 parts Silver Stain Developer) and incubated until protein bands appeared. This reaction was quenched with 5% acetic acid, washed once quickly, and incubated in 5% acetic acid for 10 min.

SARS-CoV-2 Neutralization. The target cells used for infection in viral neutralization assays were from a HeLa cell line stably overexpressing the SARS-CoV-2 receptor, ACE2, as well as the protease known to process SARS-CoV-2, TMPRSS2. Production of this cell line is described in detail in Rogers et al., 2020, with the addition of stable TMPRSS2 incorporation. (Rogers et al., 2020, “Isolation of Potent SARS-CoV-2 Neutralizing Antibodies and Protection from Disease in a Small Animal Model.” Science. 369 (6506), 956-963. doi.org/10.1126/science.abc7520; Crawford, et al., 2020, “Protocol and Reagents for Pseudotyping Lentiviral Particles with SARS-CoV-2 Spike Protein for Neutralization Assays.” Viruses, 12 (5).doi.org/10.3390/v12050513). ACE2/TMPRSS2/HeLa cells were plated one day prior to infection at 5,000 cells per well. White-walled, clear-bottom plates (96 wells) were used for the assay (Thermo Fisher Scientific). On the day of the assay, dilutions of serum were made into sterile D10 medium (DMEM+10% FBS, L-glutamate 5 mL, penicillin and streptomycin (5 mL of 100×), and 10 mM HEPES) to a final volume of 60 μL. For viral neutralization assays, mouse serum was centrifuged at 2000×g for 15 min, and heat inactivated for 30 min at 56° C. Samples were run in technical duplicate in each experiment. All other wells contained only D10 medium. A virus mixture was made containing the virus of interest (for example SARS-CoV-2 with a 21 amino acid deletion at the C terminus), D10 medium (DMEM+10% FBS, L-glutamate, penicillin, streptomycin, and 10 mM HEPES), and polybrene (such that the final concentration was 5 μg/mL in inhibitor/virus dilutions). Virus dilutions into medium were selected such that a suitable signal would be obtained in the virus-only wells (luminescence >10,000 RLU). Sixty microliters of this virus mixture were added to each of the inhibitor dilutions to a final volume of 120 μL in each well. Virus-only wells contained 60 μL D10 medium and 60 μL virus mixture. Cells-only wells contained 120 μL of D10 medium. The serum dilution/virus mixture was left to incubate for 1 h at 37° C. Following incubation, the medium was removed from the cells on the plates made one day prior, replaced with 100 μL of inhibitor/virus dilutions, and incubated at 37° C. for approximately 48 h. Infectivity readout was performed by measuring luciferase levels 48 h post-infection. 50 μL of medium were removed from all cells and then cells were lysed by the addition of 50 μL BriteLite™ assay readout solution (Perkin Elmer) into each well. Luminescence values were measured using a BioTek Synergy™ HT Microplate Reader (BioTek). Each plate was normalized by averaging cells-only (0% infectivity) and virus-only (100% infectivity) wells. Normalized values were fit with a three-parameter non-linear regression inhibitor curve in GraphPad Prism 9.1.0 to obtain IC₅₀ values.

SARS-CoV-2 Spike Pseudotyped Lentivirus Production. Viral transfections were done in HEK293T cells using calcium phosphate transfection reagent. Six million cells were seeded in D10 medium (DMEM+10/a FBS, L-glutamate, penicillin, streptomycin, and 10 mM HEPES) in 10-cm plates one day prior to transfection. A five-plasmid system was used for viral production, as described in Crawford et al., 2020, “Protocol and Reagents for Pseudotyping Lentiviral Particles with SARS-CoV-2 Spike Protein for Neutralization Assays.” Viruses, 12 (5). doi.org/10.3390/v12050513. The Spike vector contained the 21 amino acid truncated form of the SARS-CoV-2 Spike sequence from the Wuhan-Hu-1 strain of SARS-CoV-2 (Genebank: BCN86353.1). The plasmids were added to D10 medium in the following amounts: 10 μg pHAGE-Luc2-IRS-ZsGreen, 3.4 μg FL Spike, 2.2 μg HDM-Hgpm2, 2.2 μg HDM-Tat1b, 2.2 μg pRC-CMV-Rev1b in a final volume of 1 mL; subsequently, 30 μL of Bio T were added. Transfection reactions were incubated for 10 min at room temperature, and then filled to 10 mL with D10 medium. These samples were then added slowly to plated cells without medium. After 24 h (post-transfection), medium was removed and replaced with fresh D10 medium. Viral supernatants were harvested 72 h post-transfection by spinning at 300×g for 5 min followed by filtering through a 0.45 μm filter. Viral stocks were aliquoted and stored at −80° C. until further use.

It is understood that the examples and embodiments described in the present disclosure are for illustrative purposes only and that various modifications or changes in light thereof will be suggested to persons skilled in the art and are to be included within the spirit and purview of this application and scope of the appended claims. All publications, patents, and patent applications cited in the present disclosure are hereby incorporated by reference in their entirety for all purposes.

1. A composition comprising an isolated Staphylococcus aureus (S. aureus) peptidoglycan (PGN) sacculus bonded to an immunogenic polypeptide via a triazole moiety.

2. The composition of claim 1, wherein the triazole moiety is a reaction product between an azide-modified D-amino acid on the S. aureus PGN sacculus and a cycloalkyne moiety bonded to the immunogenic polypeptide via a linker moiety.

3. The composition of claim 2, wherein the azide-modified D-amino acid is azido-D-alanine (azaDala).

4. The composition of claim 2, wherein the linker moiety is a reaction product between an amino acid residue in the immunogenic polypeptide and a crosslinker reagent comprising the cycloalkyne moiety and a peptide-reactive handle.

5. The composition of claim 4, wherein the cycloalkyne moiety is a cyclooctyne moiety.

6. The composition of claim 5, wherein the cyclooctyne moiety is dibenzocyclooctyne (DBCO).

7. The composition of claim 4, wherein the peptide-reactive handle is a maleimide and the amino acid residue in the immunogenic polypeptide is a cysteine residue.

8. The composition of claim 4, wherein the peptide-reactive handle is a N-hydroxysuccinimide moiety.

9. The composition of claim 4, wherein the crosslinker reagent further comprising one or more ethylene glycol moieties.

10-11. (canceled)

12. The composition of claim 1, wherein the S. aureus PGN sacculus is a peptidoglycan sacculus selected from S. aureus strain ATCC 25923, ATCC 29213, SH1000, RN4220, or RN4220 (ΔTarO).

13. The composition of claim 12, wherein the S. aureus PGN sacculus is a peptidoglycan sacculus from S. aureus strain SH1000.

14. The composition of claim 1, wherein the S. aureus PGN sacculus is bonded to a plurality of different immunogenic polypeptides via triazole moieties.

15. (canceled)

16. The composition of claim 1, wherein the immunogenic polypeptide is a SARS-CoV-2 Spike protein or a fragment thereof or a cancer neoantigen.

17-22. (canceled)

23. A formulation comprising the composition of claim 1 and a pharmaceutically acceptable excipient.

24. (canceled)

25. A method of inducing an immune response in a subject, the method comprising administering to the subject a therapeutically effective amount of the formulation of claim 23.

26. The method of claim 25, wherein the method elicits an antibody response and/or a T-cell response in the subject.

27. (canceled)

28. The method of claim 25, wherein the immunogenic polypeptide is a SARS-CoV-2 Spike protein or a fragment thereof, and wherein the formulation is administered in an amount capable of eliciting a protective immune response against the SARS-CoV-2 Spike protein in the subject.

29. (canceled)

30. The method of claim 25, wherein the immunogenic polypeptide is a protein expressed in a cancer cell, and wherein the formulation is administered in an amount capable of eliciting a protective immune response against a cancer.

31. (canceled)

32. The method of claim 30, wherein the subject has, has had, or is at risk of developing the cancer.

33. (canceled)

34. A kit comprising the formulation of claim 23 packaged in a container and instructions for the administration thereof.

35-38. (canceled)
